(19) United States
(12) Patent Application Publication
(10) Pub. No.: US 2026/0209433 A1
(43) Pub. Date: Jul. 23, 2026

(54) DUAL CURABLE VITRIMERS

(71) Applicant: LUXEMBOURG INSTITUTE OF SCIENCE AND TECHNOLOGY (LIST), Esch-Sur-Alzette (LU)

(72) Inventors: Pierre VERGE, Esch-Sur-Alzette (LU); Ambre MEYER, Esch-Sur-Alzette (LU); Antoine ADJAOUD, Esch-Sur-Alzette (LU); Laura PUCHOT, Esch-Sur-Alzette (LU); Alexander S. SHAPLOV, Esch-Sur-Alzette (LU); Joamin GONZALEZ GUTIERREZ, Esch-Sur-Alzette (LU); Charles JEHL, Esch-Sur-Alzette (LU)

(21) Appl. No.: 19/139,513

(22) PCT Filed: Dec. 8, 2023
(86) PCT No.: PCT/EP2023/025539

§ 371 Date: Jun. 16, 2025

(30) Foreign Application Priority Data
Dec. 16, 2022 (LU) LU503197

(51) Int. Cl. C08G 65/332; C07D 413/12

(52) U.S. Cl. CPC C08G 65/332; C07D 413/12

(57) ABSTRACT

A process for synthesizing an ester and acrylate moieties-containing benzoxazine monomer, including a) reacting a phenolic carboxylic acid, including at least one R* group on the phenolic ring with a di-hydroxyl moiety bearing compound, at a temperature of from 25° C. to 200° C., during 1 h-72 h, in the presence of a catalyst of Bronsted acid type and under inert atmospheric conditions, resulting in a phenol terminated oligomer or molecule, and b) reacting the phenol terminated oligomer or molecule with a mixture of an amino-alcohol of formula (V), an aldehyde compound selected from formaldehyde and paraformaldehyde and a primary amine at a temperature of from 50° C. to 200° C., during 1 h-12 h, under inert atmospheric conditions, providing a compound of formula (VII), and reacting the compound obtained in step b) with a compound bearing acrylate or methacrylate groups.

### TECHNICAL FIELD

The invention is directed to the field of dual curable benzoxazine derivatives vitrimers and to a process of manufacturing thereof and the use of said vitrimers in various applications.

### BACKGROUND

Vitrimers are polymeric materials owing to their dynamic nature of the covalent network, arising from reversible chemical bonds, allowing the material to be healed, recycled and reprocessed like thermoplastics. These exchange reactions are triggered by external stimulus, most frequently temperature. The viscosity of vitrimers gradually decreased upon heating providing malleability to the network while permitting internal stress to relax. Network integrity over the entire range of application ensures mechanical and solvent resistance.

Following the prototypal vitrimer developed by Leibler et al. in 2011 (D. Montarnal, M. Capelot, F. Toumilhac and L. Leibler, Science, 2011, 334, 965-968), dynamic transesterification reactions demonstrated extensive interest over the last decade. These chemical exchanges induced at elevated temperatures between ester linkages and hydroxyl groups are responsible for topology rearrangements. Transesterification mechanism was implemented in cross-linked network to design self-healable, recyclable and reprocessable material with tunable properties.

Demongeot et al. (A. Demongeot, R. Groote, H. Goossens, T. Hoeks, F. Tournilhac and L. Leibler, Macromolecules, 2017, 50 (16), 6117-6127) adapted the vitrimer concept to commercially available thermoplastic. Cross-linked polybutylene terephthalate (PBT) vitrimer based on transesterification exchanges was successfully prepared by reactive extrusion. In addition to improving the manufacturing techniques and the potential scope of these networks, global environmental context urges the scientific community to promote sustainable polymer derived from naturally occurring feedstocks. Altuna et al. (F. I. Altuna, V. Pettarin and R. Williams, Green Chem., 2013, 15, 3360-3366) endeavored to generate fully bio-based polyester showing properties reminiscent of vitrimers, starting from epoxidized soybean oil and an aqueous citric acid solution. Furthermore, Legrand et al. (A. Legrand and C. Soulié-Ziakovic, Macromolecules, 2016, 49, 5893-5902) enabled to extend the scalability of applications of vitrimer networks by developing a silica-reinforced epoxy vitrimer nanocomposites with enhanced properties.

Polybenzoxazines are a new type of thermoset with outstanding mechanical and thermal properties. As many other thermosets, they cannot be reshaped, re-processed nor recycled. A few examples have been reported showing a reasonable level of healability (L. Zhang, Z. Zhao, Z. Dai, L. Xu, F. Fu, T. Endo, X. Liu, ACS Macro. Lett. 2019, 8, 5, 506-511 and Arslan M., Kiskan B., Y. Yagci, Sci. Rep. 2017, 7, 5207).

The Applicant has shown that some various chemical structures of polybenzoxazine type vitrimers exhibit among others self-healing, reshaping, reprocessability, high strength, and low melt viscosities properties owing to benzoxazine moieties of the starting monomers used for producing corresponding vitrimers, through polymerization thereof. WO 2021/180562 A1 relates to vitrimers obtained through the polymerization of disulfide-containing benzoxazine monomers. WO 2022/122735 A1 and WO 2021/250024 relate to vitrimers obtained through the polymerization of ester containing benzoxazine monomers.

It appears that vitrimers, especially those originating from benzoxazine monomers, in particular ester-containing benzoxazine monomers, still need to present some improved properties for specific uses. In some instances, said monomers exhibit some 3D printing abilities but they need to be heated, after said 3D printing step, to obtain the corresponding 3D shaped vitrimers. The main drawback of this heating step is that the 3D shaped structure is lost, i.e. polybenzoxazine vitrimers of 3D printed structure cannot be obtained. Besides, there is a need of some vitrimers exhibiting lower viscosities at room temperature, higher Tg, higher mechanical properties, with comparison to those already existing.

### SUMMARY

The invention obviates one of the previous drawbacks, and relates to an ester and acrylate moieties containing benzoxazine monomer of formula (I),

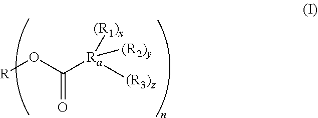

wherein
R₁ is

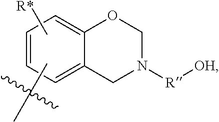

R₂ is

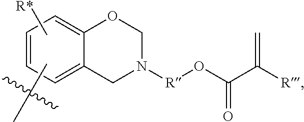

and
R₃ is

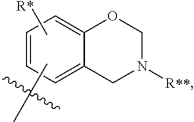

wherein
Ra is selected from the group consisting of a linear or branched C₁-C₆ alkyl or alkoxy group, a linear or branched C₂-C₆ alkenyl or alkylenoxy group, a substituted or unsubstituted linear or branched C₂-C₆ alkynyl group, and a —C-linear or branched C₁-C₆ alkyl or C₂-C₆ alkenyl substituted or unsubstituted phenyl group;
R* is selected from the group consisting of H, OH and a O-linear or branched C₁-C₈alkyl group, a linear or branched C₁-C₁₅ alkyl group or a C₂-C₁₅ alkenyl group or

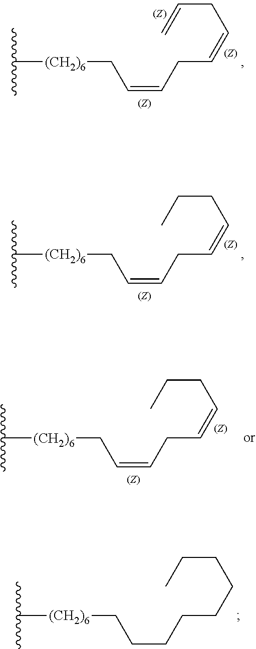

R″, R** and R are, independently, selected from the group consisting of a linear or branched C₁-C₆ alkyl or alkoxy group; a linear or branched C₂-C₆ alkenyl or alkylenoxy group; a substituted or unsubstituted linear or branched C₂-C₆ alkynyl group; at least one linear or branched C₁-C₆ alkyl or C₂-C₆ alkenyl substituted or unsubstituted o-, m-, p-phenyl group, cyclo(C₃-C₆alkyl) group or a heterocyclo(C₃-C₆alkyl) group, wherein the hetero atom is selected from N, S, and O; a —(CH₂)ₙ₃-phenyl group, a —(CH₂)ₙ₁—O—(CH₂)ₙ₂ group, a —CHₙ₁((CHₙ₂OH)ₙ₁)CHₙ₁— group wherein n1 and n2, independently, are an integer from 1 to 10,

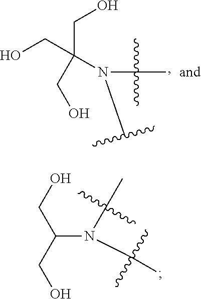

R′″ is Me, H or CN;
n3 is an integer 1-10;
with the proviso that
1≤n<50 and x>0, y>0 and z≥0, and, independently of n, x+y+z=1;
x, y, z represent the proportion between benzoxazine groups when prepared from an aminoalcohol and the other amine(s) and methacrylate groups,

$$x = \frac{{n}_{aminoalcohol}}{{n}_{functional groups}^{total}}$$

$$y = \frac{{n}_{acrylate}}{{n}_{functional groups}}$$

$$z = \frac{{n}_{amines}}{{n}_{functional groups}}$$

wherein

$${n}_{functional groups}^{total} = {n}_{aminoalcohol} + {n}_{amines} + {n}_{acrylate} , and {n}_{aminoalcohol}$$

being the number of aminoalcohol, nₐₘᵢₙₑₛ represent the number of amines (exception the number of aminoalcohol), n_{acrylate} corresponds to the number of acrylate/methacrylate groups, and

$${n}_{funtional groups}^{total}$$

is the total number of functional groups bore by the benzoxazine rings.

The values of x and z depend on the molar ratio between the number of amines (nₐₘᵢₙₑₛ) and aminoalcohol (n_{alcohol}) used to make the benzoxazine rings. The value of y depends on the rate of conversion of aminoalcohol in acrylate/methacrylate functional groups. These x, y and z are precisely defined in the process for obtaining ester and acrylate moieties containing benzoxazine monomer of formula (I) of the invention.

The ester and acrylate moieties-containing benzoxazine monomer(s) of the invention are advantageously suited for obtaining polybenzoxazine vitrimers by a double curing procedure, i.e. involving successive curing steps, which is very advantageously a 3D printing procedure, wherein, in said polybenzoxazine vitrimers, the 3D shaped structure is maintained, by contrast to known polybenzoxazine vitrimers. This is explained by the fact that known benzoxazine monomers yielding vitrimers (i) present too high viscosities to be 3D printed, (ii) they need to be cured after printing and (iii) they are not UV curable.

The preparation of the polybenzoxazine vitrimers, originating from ester and acrylate moieties-containing benzoxazine monomer(s) of the invention, involves a double curing procedure including a first curing step by UV treatment polymerization undergoing the polymerisation of the acrylate moieties, allowing to fix the shape of the obtained structure. The first curing step is followed by a second curing step performed by a thermic treatment undergoing the polymerisation of the benzoxazine moieties of the ester and acrylate moieties-containing benzoxazine monomer(s), which do not melt during said thermal treatment owing to the prior UV treatment for the polymerization of the acrylate moieties.

In the context of the invention, “polybenzoxazine vitrimers”, “dual cured vitrimers”, have the same meaning.

The advantage is that the preparation of such dual cured vitrimers may be carried out during the 3D printing procedure or even, in some embodiments, after the 3D printing procedure.

The benzoxazine ring opening and a self-polymerisation in the ester and acrylate moieties-containing benzoxazine monomer(s) of the invention, performed by or during the second curing step under heat, result in said polybenzoxazine vitrimers. Said specific monomer(s), as starting product(s), exhibit low viscosity at 25° C., typically 100-10000 mPa·s, better 3000-8000 mPa·s. For the rest of the document, benzoxazine vitrimers will always refer to the polymerized form of the ester-bond benzoxazine monomers.

The dual cured vitrimers properties are tightly connected to the properties of the ester-containing benzoxazine monomer.

As may be seen from formula (I), the monomer(s) includes a benzoxazine ring moiety that allows the cross-linking of said monomer upon heating and that promotes the reprocessing of the obtained benzoxazine vitrimers thanks to the exchangeable ester bonds it forms once crosslinked. Benzoxazine gives thermosetting properties such as high-temperature and flammability performance, high strength, thermal stability, low water absorption, chemical resistance, low melt viscosities, and near-zero shrinkage.

The presence of a moiety consisting in ester bonds, free aliphatic hydroxyl groups and acrylate moieties are essential to form a dynamic network of the benzoxazine vitrimers, allowing the material to be recycled, reshaped and reprocessed. An amine terminated with a hydroxyl group allows to close the oxazine ring and allows the transesterification reactions. Accordingly, the essential features of the monomer(s) of the invention rely on the benzoxazine-containing moiety, ester bonds, free aliphatic hydroxyl groups and acrylate moieties. The Tg of such polybenzoxazine vitrimers may be of from −50° C. to 250° C., and the elastic modulus in the range 0.1-4 GPa, said elastic modulus being measured by classical thermomechanical analysis.

Preferentially, n is within the range of 1-10, more preferably within the range of 1-5, and independently Ra may be selected from the group consisting of a linear or branched C₁-C₄ alkyl or alkoxy group, a linear or branched C₂-C₄ alkenyl or alkylenoxy group, a substituted or unsubstituted linear or branched C₂-C₄ alkynyl group, and a linear or branched C₁-C₄ alkyl or C₂-C₆ alkenyl substituted or unsubstituted phenyl group; and, independently,
R* may be selected from the group consisting of H, OH and a O-linear or branched C₁-C₄ alkyl group, a linear or branched C₁-C₁₀ alkyl group or a C₂-C₁₀ alkenyl group, more preferentially a linear or branched C₁-C₆ alkyl group, a C₂-C₆ alkenyl group or a C₂-C₆ alkynyl group or

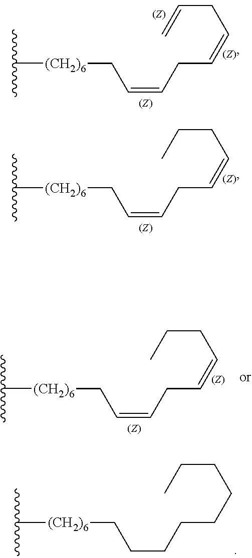

Preferably, in the definition of R″, each C atom, independently, of the phenyl group, the cyclo(C₃-C₆alkyl) group or the heterocyclo(C₃-C₆alkyl) group may bear substituents as defined above.

Besides, the ester and acrylate moieties-containing benzoxazine monomer(s) of the invention may be used in combination or mixed with another benzoxazine monomer such as selected from the group consisting of monofunctional amines bridged with diphenolic compounds, monophenolic compounds bridged with diamines and diamines bridged by diphenolic compounds, or a mixture thereof, to improve the processability of the ester and acrylate moieties-containing benzoxazine monomer(s) during the polymerization for obtaining a vitrimer thereof (improvement of the viscosity of the obtained mixture: monomer of the invention, monomer 1, and said other monomer, monomer 2). The polybenzoxazine derivatives vitrimers properties are tightly connected to the properties of the ester and acrylate moieties-containing benzoxazine monomer(s). The mixture of both monomers 1 & 2 may be in the ratio monomer1:monomer 2 of 1 wt %-90 wt %.

The invention also related to a process for synthesizing an ester and acrylate moieties-containing benzoxazine monomer of formula (I), comprising the following steps consisting of:
a) reacting a phenolic carboxylic acid of formula (II), comprising at least one R* group on the phenolic ring:

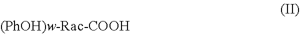

wherein
Rac has the definition of Ra and w=1, 2 or 3;
with a hydroxyl moiety bearing compound of formula (III)

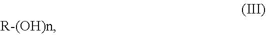

wherein R has the definition of R″ and n has the same definition than above,
at a temperature of from 25° C. to 200° C., during 1 h-72 h, in the presence of a catalyst of Bronsted acid type and under inert atmospheric conditions, resulting in a phenol terminated oligomer or molecule (compound (IV),
   b) reacting the compound (IV) with a mixture of:
      an amino-alcohol of formula (V):

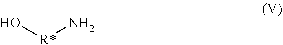

an aldehyde compound selected from formaldehyde and paraformaldehyde of CH₂O formula

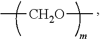

where m is an integer of from 8 to 100,
a primary amine of formula (VI),

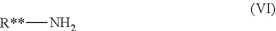

at a temperature of from 50° C. to 200° C., during 1 h-12 h, under inert atmospheric conditions, providing a compound of formula (VII), and
c) reacting the compound (VII) with a compound bearing acrylate or methacrylate groups, said compound being at least one defined by compounds (VIII)-(XII), (meth)acrylic acid chloride of formula (VIII)

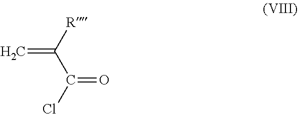

in polar solvents, at temperatures from −5° C. to 25° C.; or methyl(meth)acrylate of formula (IX)

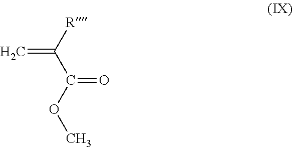

in the presence of a catalyst and a polymerization inhibitor; or (meth)acrylic acid of formula (X)

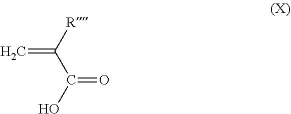

in the presence of a catalyst and an azodicarboxylate in a solvent at temperatures from −10° C. to reflux; or
an excess of (meth)acrylic acid of formula (X) in the presence of a catalyst; or
(meth)acrylic acid anhydride of formula (XI) or asymmetric anhydride of formula (XII)

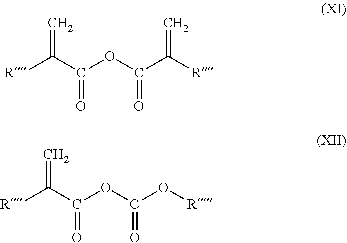

in the presence of a catalyst;
wherein
   R″″ is H or CH₃.
   R′″″ is CH₃ or C₂H₅;
at temperatures of from −10° C.-50° C., during 12 h-48 h, for obtaining the ester and acrylate moieties-containing benzoxazine monomer of formula (I),
x, y, z, in formula (I), represent the proportion between benzoxazine groups when prepared from an aminoalcohol and the other amine(s) and methacrylate groups, the latter obtained after step c),

$$x = \frac{{n}_{aminoalcohol}}{{n}_{functional groups}^{total}}$$

$$y = \frac{{n}_{acrylate}}{{n}_{functional groups}}$$

$$z = \frac{{n}_{amines}}{{n}_{functional groups}}$$

wherein

$${n}_{functional groups}^{total} = {n}_{aminoalcohol} + {n}_{amines} + {n}_{acrylate} , and {n}_{aminoalcohol}$$

being the number of aminoalcohol, nₐₘᵢₙₑₛ represent the number of amines (exception the number of aminoalcohol), n_{acrylate} corresponds to the number of acrylate/methacrylate groups, and

$${n}_{funtional groups}^{total}$$

is the total number of functional groups bore by the benzoxazine rings,
with the proviso that when at least one R* of the phenolic acid derivative is in ortho position with regard to —OH group, then R* is H.

In the process, R, Ra, R*, R** and R″ have the definition given for formula (I).

The values of x and z depend on the molar ratio between the number of amines (nₐₘᵢₙₑₛ) and aminoalcohol (n_{alcohol}) used to make the benzoxazine rings. The value of y depends on the rate of conversion of aminoalcohol in acrylate/methacrylate functional groups.

According to the process, the “z” value could be 0 (z=0), independently of the “x” value, meaning that the primary amine may, in some embodiments, be omitted. The compound of formula (V), the amino-alcohol, is used for both.

The Applicant has shown that the specific starting reactants are providing a ester and acrylate moieties-containing benzoxazine monomer, which in turn, after polymerization, is giving the polybenzoxazine derivatives vitrimers comprising polymerized benzoxazine.

No reactive diluents and no solvents are required for steps a) and b). Other advantages of the obtained monomer(s) are those mentioned above, especially exhibiting low viscosity at 25° C., typically 1000-10000 mPa·s, better 300-800 mPa·s, high Tg values.

The step a) may advantageously be carried out at a temperature in the range of 80° C. to 170° C., most preferably of from 100° C. to 140° C. for the best synthesis yields of higher than 95%, the chosen temperature being dependent on the nature of the reactants, i.e. the melting temperature of said reactant medium.

Advantageously, step a) is performed of from 12 h to 48 h, better of from 12 h to 36 h, for the highest yield of at least 95%, and the duration is based on the kinetic of the reaction.

The Bronsted acid type catalyst are those commonly used for a Fischer esterification include para-toluene sulfonic acid (p-TSA), anhydrous chlorohydric acid (HCl), phosphoric acid (H₃PO₄), methanoic acid (CH₃—CO₂H), sulfuric acid, tosylic acid, and Lewis acids such as scandium(II) triflate. The content of catalyst may typically be of from 0.5 wt % to 2 wt %.

The respective stoichiometry of starting reactants on step a), phenolic carboxylic acid:hydroxyl moiety bearing compound may preferably be n eq.: 1.0 eq, resulting in an 1.0 eq. of the phenol terminated oligomer or molecule (compound (IV).

Step a) may be performed under inert atmospheric conditions, such as by the use of Ar or N gas.

The second step of the process, step b), corresponds to a Mannich condensation type reaction of the phenol terminated oligomer or molecule of step a) ((IV)) with the amino-alcohol (formula (V)), the aldehyde compound selected from formaldehyde and paraformaldehyde and the primary amine of formula (VI), optionally in presence of a catalyst. Thus, since step b) does not require the use of an external catalyst, step b) is implemented in an easier way. Step b) allows the formation of R₁ and R₃.

Step b) may be performed under inert atmospheric conditions, such as by the use of Ar or N gas.

Advantageously, the amino-alcohol of formula (V) includes R* group, a linear amino-alcohol with a primary amine moiety and an aliphatic hydroxyl moiety for obtaining with the highest yield and the best reaction conditions the oxazine ring.

The amino-alcohol of formula (V) may be more preferably selected from the group consisting of 2-aminoethanol, 2-amino-2-methylpropanol, 5-aminopentan-1-ol, heptaminol, 2-(2-Aminoethoxy)ethanol, and diglycolamine, or mixtures thereof.

The primary amine includes the R** group as defined above.

Primary amines may be further selected from the group consisting in allylamine, methylamine, ethylamine, propylamine, butylamine, isopropylamine, hexylamine, cyclohexylamine, stearylamine, 2-aminofluorene, aminophenyl acetylene, propargyl ether aniline, 4-aminobenzonitrile, furfurylamine and aniline, or mixtures thereof.

The respective stoichiometry of starting reactants on step b), phenol terminated oligomer or molecule (IV):amino-alcohol (V):primary amine (VI):paraformaldehyde may preferably be 1.0 eq.: n(x+y): nz: 2.0 n (x+y+z) resulting in an 1.0 eq. of compound (VII). In some further examples, said stoichiometry may be 1.0 eq.:2.0 eq.: 0.0 eq.: 4.0 eq resulting in an 1.0 eq. of compound (VII).

The specific range stoichiometry is depending on the respective equivalent proportion of the amino-alcohol and of the primary amine. It should be pointed out that there is a minimal quantity required for the reaction to occur. For instance, the relative molar % of amino-alcohol vs the relative molar % of primary amine derivative is 10 molar % vs 90 molar % respectively. It also means that primary amine may be omitted (0 molar %) and amino-alcohol may only be used instead (100 molar %). Besides, the selected stoichiometry ranges of both amino-alcohol/amine and paraformaldehyde preferably avoids the formation of either reaction linear and/or aliphatic by-products, such as oxazolidine, triaza derivatives, or condensation derivatives.

The temperature range of step b) may preferably be of from 70° C. to 150° C., more preferably, of from 70° C. to 120° C., allowing to obtain the highest conversion yields of at least 75%.

Advantageously, step b) is performed from 1 h to 8 h, preferably of from 1 h to 5 h, for the highest yield of at least 75%.

The third step of the process, step c), is the reaction of the compound (VII) of step b) with a compound bearing acrylate and/or methacrylate groups, the latter compound being at least one defined by compounds (VIII)-(XII), or mixture thereof.

In an embodiment, step c) may be performed with compound (VIII) -(meth)acrylic acid chloride- typically via a classical acylation reaction with compound (VII) of step b), in the absence of any suitable catalyst or in the presence of a catalyst/hydrogen absorbent, preferably selected from, and not limited to, triethyl amine (TEA), pyridine, N,N-Diisopropylethylamine (DIPEA) and N−N-dimethylaniline, in polar solvents, selected from, and not limited to, acetonitrile, chloroform, dichloromethane and N,N-dimethylformamide (DMF), preferably at temperatures of from −5° C. to 15° C., for 12 h-48 h.

According to another embodiment, step c) may be performed with compound (IX) -methyl(meth)acrylate- typically via a classical re-esterification reaction, in the presence of a catalyst, such as sodium or magnesium alcoholate, and a polymerization inhibitor, such as p-oxydiphenylamine, with a preferable simultaneous distillation of the resulting azeotropic mixture, which is for example consisting of methanol with methyl ether, for 12 h-48 h.

Step c) may be carried with compound (X) -(meth)acrylic acid- typically via a classical Mitsunobu reaction involving the presence of a catalyst, such as triphenylphosphine, and an azodicarboxylate, such as diethyl azodicarboxylate (DEAD) or diisopropyl azodicarboxylate (DIAD), in a solvent, such as tetrahydrofuran (THF) or toluene, at temperatures from −10° C. to reflux, for 12 h-48 h.

According to an alternate embodiment using compound (X) -(meth)acrylic acid-, step c) may be performed with an excess of said (meth)acrylic acid, via a classical esterification reaction in the presence of an acidic catalyst, being preferably selected from p-toluenesulfonic acid (p-TCA), sulfuric acid (H2SO4) and methanesulfionic acid (CF3SO3H), with simultaneous distillation of the resulting azeotropic mixture, for example consisting of toluene or cyclohexane with water, for 12 h-48 h.

Still according to another embodiment, step c) may be performed with compound (XI) -(meth)acrylic acid anhydride- or with compound (XII) -(meth)acrylic acid asymmetric anhydride-, via a classical acylation reaction in the presence of a catalyst, being preferably selected from p-toluenesulfonic acid (p-TCA), sulfuric acid (H2SO4) and anhydrous ZnCl2, at temperature range of −5° C.-15° C.

Step c) allows the obtention of R2.

Advantageously, the whole synthesis may generally not require any further monomer purification for the invention to be implemented. However, the purification of the monomer, if needed, such as for example during step c) and/or after it, may be performed by any known technic (vacuum, distillation, resolubilisation, extraction with water, with a base, like NaOH, or brine, etc.). Solvents when used may also be removed, upon need by classical implementations, such as using low pressure removal apparatus.

The reaction mixtures of both steps a) and b) are stirred using a classical mechanical stirrer, or any non-limitative means.

The process may be implemented by any known means known to the one skilled in the art, using appropriate vessel either at lab scale or at industrial scale.

The respective stoichiometry of starting reactants on step c), compound VII: compound bearing acrylate or methacrylate groups (VIII-XII) may preferably be 1.0 eq.:3.0 ny eq. resulting in an 1.0 eq. of the ester and acrylate moieties-containing benzoxazine monomer. In some embodiments, “ny” values may be of from 0.1 to 10, more preferably of from 0.5 to 2.

The invention also relates to a process for preparing a polybenzoxazine derivative vitrimer comprising a first curing step by UV treatment polymerization of an ester and acrylate moieties-containing benzoxazine monomer of the invention (formula (I)) or as obtainable by the above mentioned process, followed by a second curing step performed by a thermic treatment undergoing the polymerisation of the benzoxazine moieties of the ester and acrylate moieties-containing benzoxazine monomer(s), In the context of the invention “derivative” means that the obtained vitrimer is obtained and derived through the polymerization of the benzoxazine monomer of the invention. Accordingly, “polybenzoxazine derivative vitrimer” or “polybenzoxazine vitrimer” have the same meaning.

The preparation of the polybenzoxazine vitrimers, originating from ester and acrylate moieties-containing benzoxazine monomer(s) of the invention, involves a double curing procedure including a first curing step by UV treatment polymerization undergoing the polymerisation of the acrylate moieties of the compound of formula (I), allowing to fix the shape of the obtained structure. The first curing step is followed by a second curing step performed by a thermic treatment undergoing the polymerisation of the benzoxazine moieties of the ester and acrylate moieties-containing benzoxazine monomer(s), which do not melt during said thermic treatment owing to the prior UV treatment for the polymerization of the acrylate moieties.

In the context of the invention, “polybenzoxazine vitrimers”, “dual cured vitrimers”, have the same meaning.

The advantage is that the preparation of such dual cured vitrimers may be carried out during the 3D printing procedure or even, in some embodiments, after the 3D printing procedure.

The benzoxazine ring opening and a self-polymerisation in the ester and acrylate moieties-containing benzoxazine monomer(s) of the invention, performed by or during the second curing step under heat, result in said polybenzoxazine vitrimers.

The first curing step is carried out with the classical presence of a radical initiator, at % weights (% wt) that may be of from 1 wt % to 5 wt %, using classical devices for performing said first step (UV curing) at typical wavelengths of from 30 nm to 500 nm, preferentially of from 280 to 450 nm.

The second curing step is carried out by thermally triggered ring opening polymerization of benzoxazines, at temperature ranges from 100° C. to 250° C., preferentially of from 120° C. to 200° C. for 1 h-10 h.

The polymerization duration is depending on the curing temperature (second curing step) and/or on the nature of the single ester-containing benzoxazine monomer. The polymerization temperature is selected for a given monomer to be higher than the temperature needed to synthesize the monomer. Generally, the higher the polymerization temperature, the shorter the curing duration. For example, when the temperature of the polymerization is 250° C., the curing duration may be of at least 1 h, and for a polymerization temperature of 100° C., the curing duration may be of no more than 10 h. Preferably, the curing temperature may be of from 140° C. to 200° C., more preferably of from 120° C. to 180° C., the latter range providing curing duration of from 1.5 h to 3 h, preferably of from 1.5 h to 2.5 h. The polymerization may be performed by any known heating means, such as forced or natural convection, conduction, laser beam and infrared beam.

The process may also include a post-polymerization step consisting of a heating step which may preferably be carried out at higher temperature than that the polymerization heating step.

The invention is also directed to a polybenzoxazine derivative vitrimer, that may be obtained by the above depicted process, exhibiting at least one of the following characteristics:
(i) Tᵥ values of from 100° C. to 250° C.; preferably of from 130° C. to 220° C., more preferably of from 130° C. to 190° C., and
(ii) Relaxation temperature values, ≥Tᵥ values, of from 100° C. to 300° C., preferably of from 130° C. to 200° C., more preferably of from 130° C. to 180° C.

The vitrimers Tᵥ values are generally dependent from the nature and the content of the catalyst of step b), when present.

The relaxation temperatures typically correspond to the relaxation temperatures of the vitrimers after the appliance of a strain, for example a physical deformation such as a torsion, without the observation of vitrimers degradation.

Advantageously, the vitrimers may also exhibit at least one of the following characteristics selected from the group consisting of:
a relaxation time of from 0.5 s to 2 h, preferably of from 1 s to 1 h, more preferably of from 5 s to 50 min. The relaxation time is conventionally defined as the time for the sample to relax to a value corresponding 1/e (0,37) of its original modulus. Generally, the higher is the temperature, the shorter is the relaxation time. For example, the relaxation time is about 150 min-200 s at temperatures values of 120° C.-170° C., and of 200, preferably 100 s-20 s, at temperature ranges of 150° C. to 200° C.
   In some embodiments, the vitrimer may be deformed between 0.1% to 100% of its initial size;
an activation energy related to relaxation times may be of from 50 kJ/mol to 200 kJ/mol, preferably of from 70 kJ/mol to 170 kJ/mol, more preferably of from 100 kJ/mol to 160 kJ/mol; and
a processing temperature may be of from 100° C. to 250° C., preferably of from 130° C. to 250° C., more preferably of from 150° C. to 200° C., most preferably of from 150° C. to 170° C.

The vitrimers according to the invention may also very preferably exhibit the characteristics of behaving as a thermoset and/or an insolubility in many solvents, without been limited, such as water, CHCl₃, CH₂Cl₂, DMF, THF, aromatic solvents, such as toluene and/or xylene, ketones, alcohols or carboxylic acids. Swelling properties are observed as an extent of from 0 to 500% of the initial weight thereof. Swelling experiments may be carried out in various solvents, for example in acetone, chloroform and water to assess the formation of a cross-linked network.

Among them, chloroform is the solvent in which the vitrimer shows the highest swelling ratio of about 100%. In acetone and water, the vitrimers swell of 40%-50% and 20%-30%, respectively.

The vitrimers of the invention present self-healing, reshaping, reprocessability, recycling and reversible adhesive properties.

The vitrimers may constitute an intermediate layer between at least two substrates, such as metal, polymer, glass and ceramic material. The resulting composite material may be prepared by setting at least one ester-containing benzoxazine monomer between the two considered substrates then curing at a temperature providing the vitrimer without altering the integrity of the substrates. Each substrate may be different from the other.

Metallic substrates are not limited, and may be of aluminium, iron, steel and the like.

Polymer substrates may be of polycarbonate, acrylic, polyamide, polyethylene or terephthalate.

Benzoxazine vitrimers may then be advantageously used in non-limited various fields of technologies, such electronics, aerospace, defense and automotive fields.

The invention also relates to a composition A comprising:
a) an ester and acrylate moieties-containing benzoxazine monomer(s) of formula (I), and
b) at least one or more additional compounds of organic molecules types containing or not benzoxazine moieties.

Preferably, the organic molecules types may be polymers containing or not benzoxazine moieties.

The additional compound may be used to enhance the properties of either the monomer or the vitrimer (i.e. viscosity, mechanical and thermal properties), or both.

Polymers may be epoxy resins, bismaleimide resins, phenolic resins or benzoxazine resins, polyurethanes, polyamides, polyolefins, polyesters, rubbers. The ester-containing benzoxazine derivative of formula I may be used in a weight ratio from 0.1 to 80% of the final composition.

The compound of formula (I) may be used to provide vitrimer properties to the above mentioned polymers (self-healing, reprocessing, etc.).

The invention also relates to a composition B comprising:
a) a ester and acrylate moieties-containing benzoxazine monomer(s) of formula (I), and
b) a material selected from the group consisting of fillers, fibers, pigments, dyes, and plasticizer.

The additional compound may be used to enhance the properties of either the monomer or the vitrimer (i.e. viscosity, mechanical and thermal properties), or both.

The additional compound could be carbon fibers, glass fibers, clays, carbon black, silica, carbon nanotubes, graphene, any known means for the thermal or the mechanical reinforcement of composites.

The invention also concerns a use of the vitrimer according to the invention as a reversible adhesive, sealant, coating or encapsulating systems for substrates selected from the group consisting of a metal, polymer, glass and ceramic material. Preferably, the metal and the polymer are as above defined.

### BRIEF DESCRIPTION OF THE DRAWINGS

The invention will be more detailed in the following examples with accompanying figures.

FIG. 1 is an example of the synthesis of a compound of formula (I), CHDO-PA-mea/meth, ester-containing benzoxazine monomer, according to Example 1.

FIG. 2 displays the ¹H NMR spectrum of CHDO-PA-mea/meth ester-containing benzoxazine monomer, according to Example 1.

FIG. 3 displays the rheological measurement of the UV curing of CHDO-PA-mea/meth ester-containing benzoxazine monomer, according to Example 2.

FIG. 4a shows a honeycomb structure of 3D printing combined with a UV treatment of CHDO-PA-mea/meth ester-containing benzoxazine monomer; followed by a curing step shown in FIG. 4b), according to Example 2.

FIG. 5 shows viscoelastic properties of CHDO-PA-mea/meth dual cured vitrimer by stress relaxation experiments, according to Example 2.

FIG. 6 is an example of the synthesis of a compound of formula (I), CHDO-PA-mea/meth, ester-containing benzoxazine monomer, according to Example 3.

FIG. 7 displays the ¹H NMR spectrum of CHDO-PA-mea/meth ester-containing benzoxazine monomer, according to Example 3.

FIG. 8 displays the rheological measurement of the UV curing of CHDO-PA-mea/meth ester-containing benzoxazine monomer according to Example 3.

FIG. 9a shows a honeycomb structure of 3D printing combined with a UV treatment of CHDO-PA-mea/meth ester-containing benzoxazine monomer, according to Example 4, followed by a curing step shown in FIG. 9b).

FIG. 10 shows viscoelastic properties of CHDO-PA-mea/meth dual cured vitrimer by stress relaxation experiments, according to Example 4.

FIG. 11 is an example of the synthesis of a compound of formula (I), CHDO-PA-mea/meth, ester-containing benzoxazine monomer, according to Example 5.

FIG. 12 displays the ¹H NMR spectrum of CHDO-PA-mea/meth ester-containing benzoxazine monomer, according to Example 5.

FIG. 13 displays the rheological measurement of the UV curing of CHDO-PA-mea/meth ester-containing benzoxazine monomer according to Example 6.

FIG. 14 is an example of the synthesis of a compound of formula (I), ETDO-PA-mea/meth, ester-containing benzoxazine monomer, according to Example 7.

FIG. 15 displays the ¹H NMR spectrum of ETDO-PA-mea/meth ester-containing benzoxazine monomer, according to Example 7.

FIG. 16 is an example of the synthesis of a compound of formula (I), CHDO-PA-mea/fa/meth, ester-containing benzoxazine monomer, according to Example 8.

FIG. 17 displays the ¹H NMR spectrum of CHDO-PA-mea/fa/meth, ester-containing benzoxazine monomer, according to Example 8.

FIG. 18 is an example of the synthesis of a compound of formula (I), CHDO-PA-meth, ester-containing benzoxazine monomer, according to the comparative Example 9 (not according to the invention).

FIG. 19 displays the ¹H NMR spectrum of CHDO-PA-meth ester-containing benzoxazine monomer, according to the comparative Example 9 (not according to the invention).

FIG. 20 displays the rheological measurement of the UV curing of CHDO-PA-meth ester-containing benzoxazine monomer according to the comparative Example 10 (not according to the invention).

### DETAILED DESCRIPTION

### Example 1

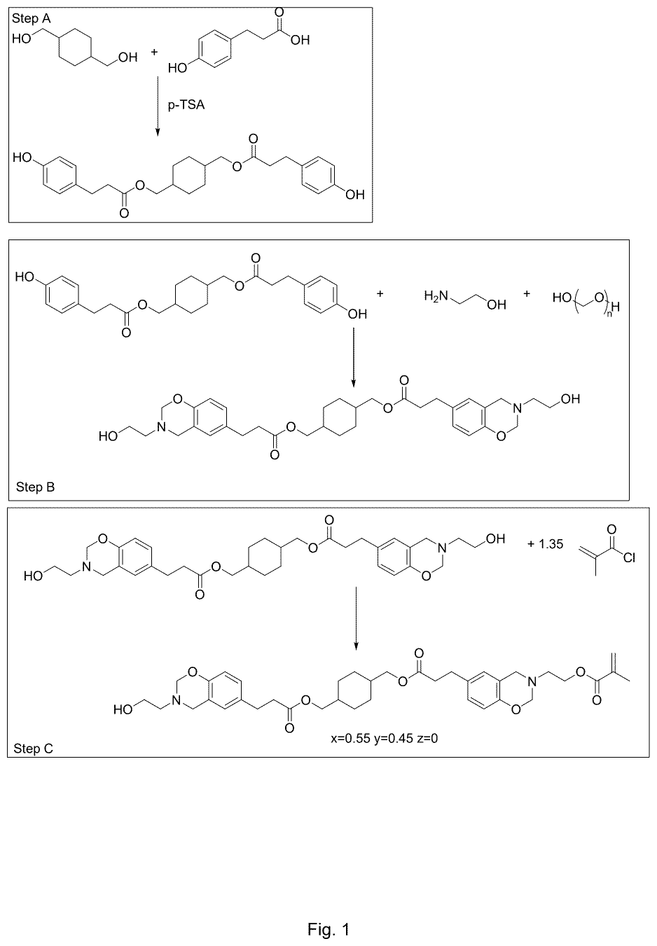

The CHDO-PA-mea/meth benzoxazine monomer containing free aliphatic hydroxyl groups and methacrylate groups was synthesized in three stages (FIG. 1). The first step, step a), corresponds to a Fischer esterification between 1,4 cyclohexanediol (cyclohexanediol, CHDO) (1 eq.) and 3-(4-hydroxyphenyl) propionic acid (phloretic acid, PA) (1 eq.) in presence of para-toluene sulfonic acid introduced in catalytic amount (0.5 wt. %). The reactants were put together in melt at 130° C. and agitated by mechanical stirring for 24 hours, to provide (CHDO-PA) (1 eq.).

The second step, step b), corresponds to a Mannich condensation of CHDO-PA (1 eq.) with mono-ethanolamine (mea) (2 eq.) and paraformaldehyde (PFA) (4 eq,). All these reactants were agitated together by mechanical stirring and reacted in melt at 70° C. for 8 hours to provide the CHDO-PA-mea benzoxazine containing free aliphatic hydroxyl groups.

In said example, the primary amine is omitted, the mono-ethanolamine (mea) is used as providing the both moieties (primary amine an amino-alcohol).

The third step, step c), corresponds to the methacrylation of CHDO-PA-mea with methacryloyl chloride (1 eq.). CHDO-PA-mea was dried overnight under reduced pressure to remove traces of water. It was thus solubilized in dried CH₂Cl₂. Triethylamine (TEA, 1 eq.) was added to the solution, as a catalyst, and the mixture was poured in a ice bath. Then, methacryloyl chloride was added drop-wise, and the solution recovered to room temperature and stirred overnight to provide CHDO-PA-mea/meth.

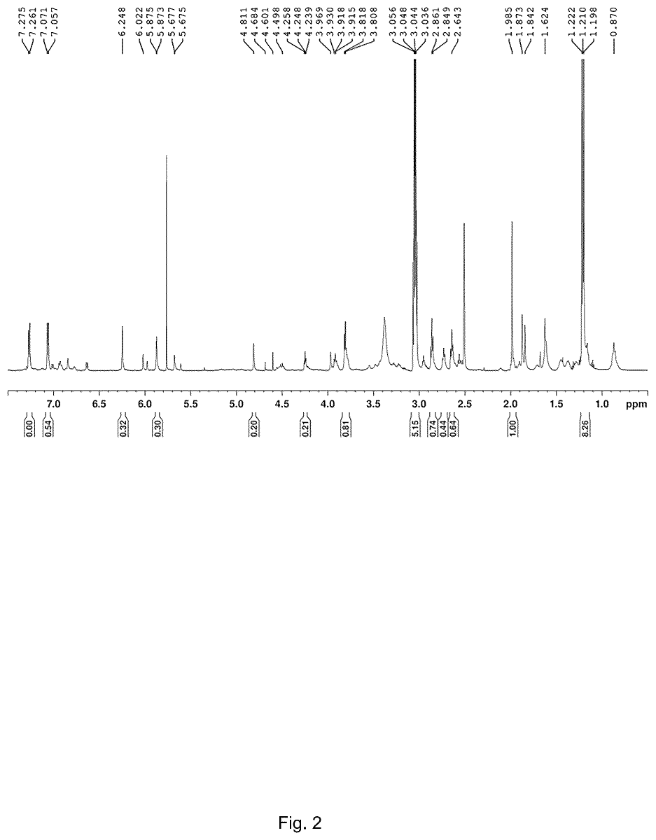

The FIG. 2 is displaying the ¹H NMR spectrum (AVANCE 111 HD Bruker spectrometer) of CHDO-PA-mea/meth ester-containing benzoxazine monomers where x=0.55, y=0.45, z=0

### Example 2. Two Step Vitrimer Synthesis from CHDO-PA-Mea/Meth

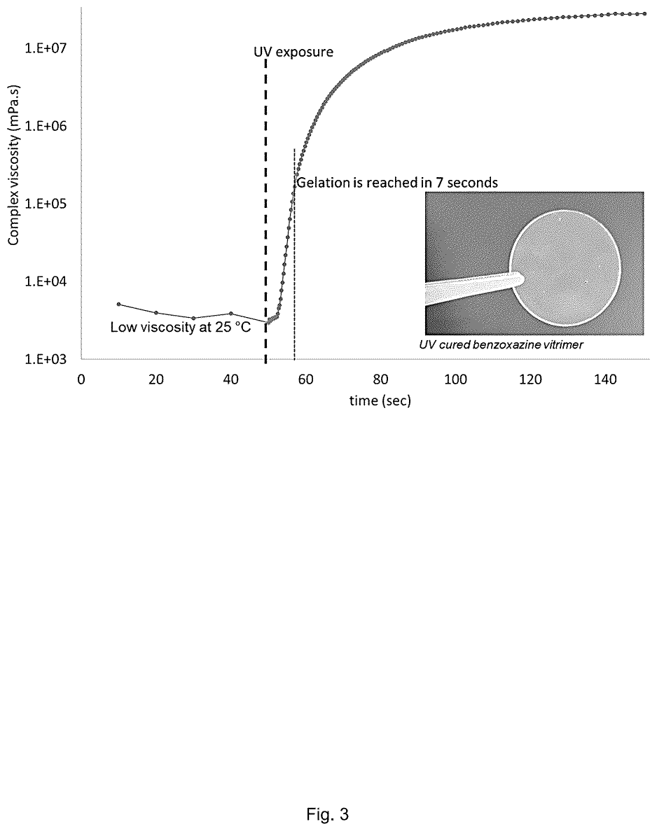

The UV curing of CHDO-PA-mea/meth of Example 1 was monitored by rheological measurement in FIG. 3. The rheogram is performed under the following conditions 1 Hz, with linear amplitude from 1 to 0.1%: 25 mm plates. The test is performed by exposing UV light with a Omnicure device (power, wavelength), the gelation is reached after 7 seconds.

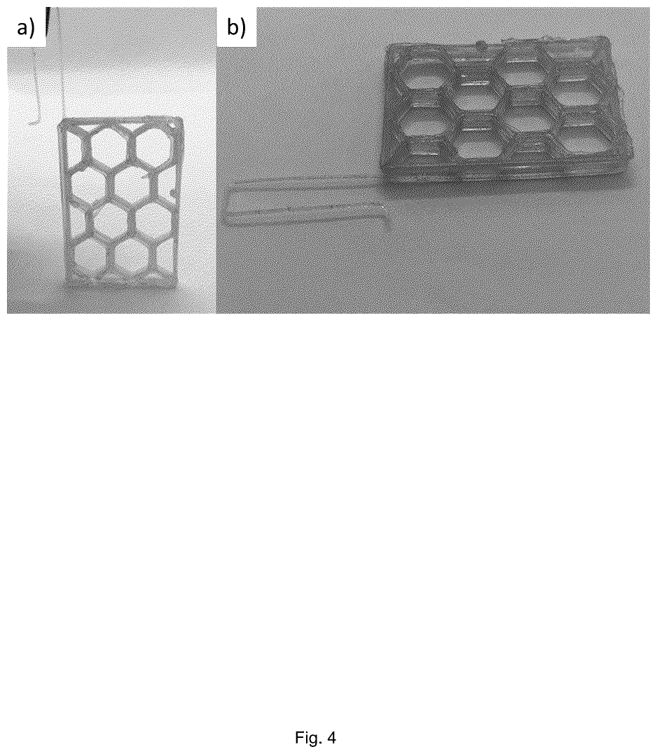

CHDO-PA-mea/meth was also 3D printed using a Hyrel System 30 M printer with an extrusion reservoir print head combined with a 356 nm UV lamp. A needle nozzle 20 gauge was used to deposit layers 0.45 mm in thickness. Extrusion occurred at room temperature on a print platform at room temperature at an extrusion speed of 1350 pulses/μm and the UV lamp continuously turned on providing the honeycomb structure in FIG. 4a. A curing step at 170° C. for 1 h was performed to consolidate the UV cured structure, leading to the material given in FIG. 4b.

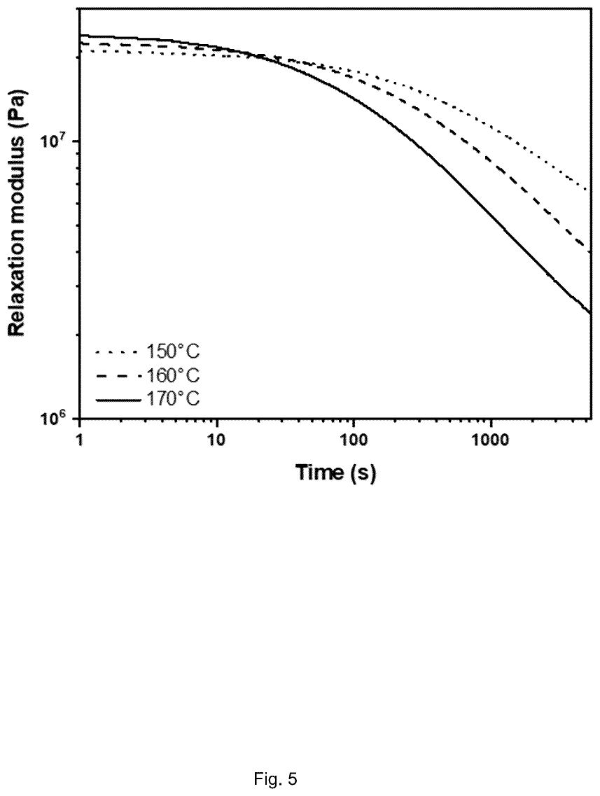

Viscoelastic properties of CHDO-PA-mea/meth dual cured vitrimer by stress relaxation experiments recorded on Anton Paar Physica MCR 302 rheometer in plate-plate mode at 1% shear strain (FIG. 5). The relaxation time of the polymer was clearly noticeable and recorded at 160° C., 170° C. and 180° C.

### Example 3: Synthesis of Benzoxazine Containing Free Aliphatic Groups, Ester Groups, and Methacrylate Groups from 1,4 Cyclohexanediol (CHDO) and 3-(4-hydroxyphenyl) Propionic Acid (PA) as Phenolic Acid Derivatives and Mono-Ethanolamine (Mea) as Primary Amine with Aliphatic OH, and Methacryloyl Chloride (MCI) as Methacrylate

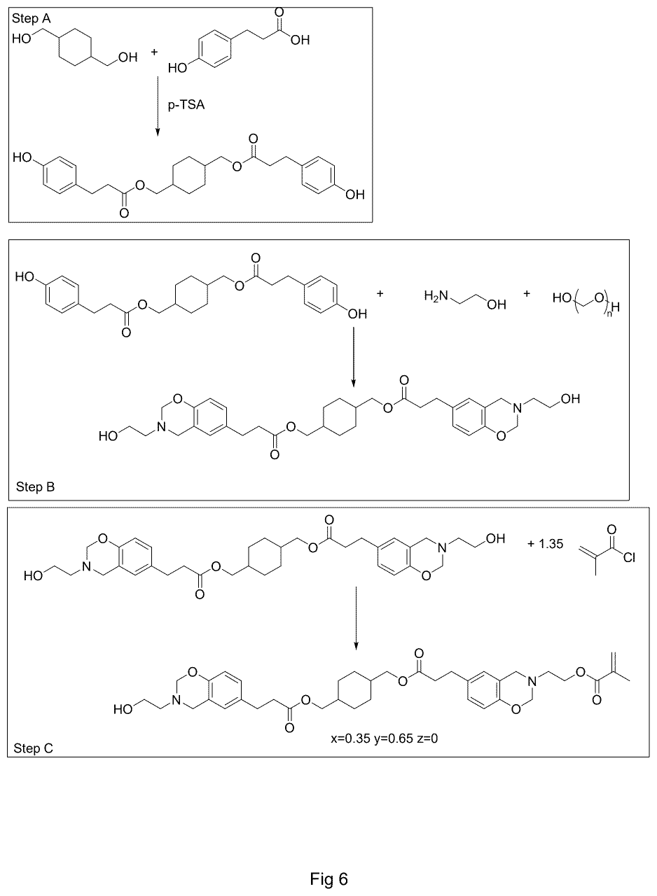

The CHDO-PA-mea/meth benzoxazine monomer containing free aliphatic hydroxyl groups and methacrylate groups was synthesized in three stages (FIG. 6). The first step, step a), corresponds to a Fischer esterification between 1,4 cyclohexanediol (cyclohexanediol, CHDO) (1 eq.) and 3-(4-hydroxyphenyl) propionic acid (phloretic acid, PA) (2 eq.) in presence of para-toluene sulfonic acid introduced in catalytic amount (0.5 wt. %). The reactants were put together in melt at 130° C. and agitated by mechanical stirring for 24 hours, to provide (CHDO-PA) (1 eq.).

The second step, step b), corresponds to a Mannich condensation of CHDO-PA (1 eq.) with mono-ethanolamine (mea) (2 eq.) and paraformaldehyde (PFA) (4 eq,). All these reactants were agitated together by mechanical stirring and reacted in melt at 70° C. for 8 hours (85° C. for 2 hours) to provide the CHDO-PA-mea benzoxazine containing free aliphatic hydroxyl groups.

In said example, the primary amine is omitted, the mono-ethanolamine (mea) is used as providing the both moieties (primary amine an amino-alcohol).

The third step, step c), corresponds to the methacrylation of CHDO-PA-mea with methacryloyl chloride (1.35 eq.). CHDO-PA-mea was dried overnight under reduced pressure to remove traces of water. It was thus solubilized in dried CH₂Cl₂. Triethylamine (TEA, 1.35 eq.) was added to the solution, as a catalyst, and the mixture was poured in a ice bath. Then, methacryloyl chloride was added drop-wise, and the solution recovered to room temperature and stirred overnight to provide CHDO-PA-mea/meth with z=0, x=0.35 and y=0.65.

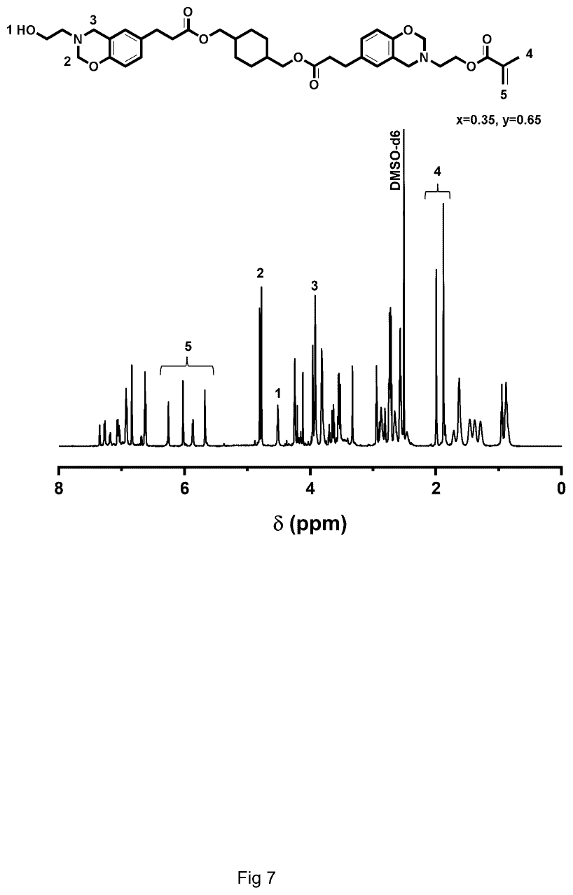

The FIG. 7 is displaying the ¹H NMR spectrum (AVANCE Ill HD Bruker spectrometer) of CHDO-PA-mea/meth ester-containing benzoxazine monomers where z=0, x=0.35 and y=0.65.

### Example 4. Two Step Synthesis from CHDO-PA-Mea/Meth with z=0, x=0.7 and v=0.3

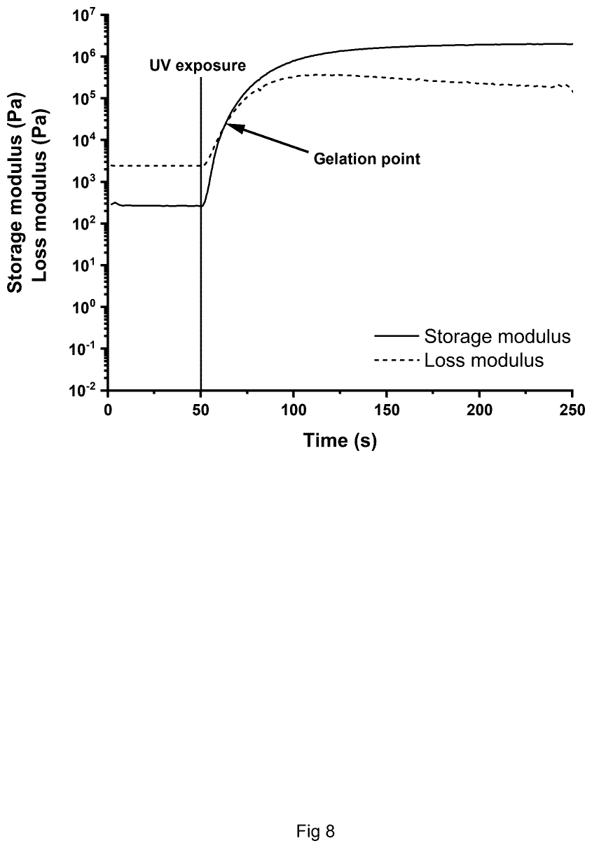

The UV curing of CHDO-PA-mea/meth of Example 3 was monitored by rheological measurement in FIG. 8. The rheogram is performed under the following conditions: 10 Hz, with constant amplitude of 0.1%; 25 mm plates. The test is performed by exposing UV light with a Omnicure device (power, wavelength), the gelation is reached after 13 seconds.

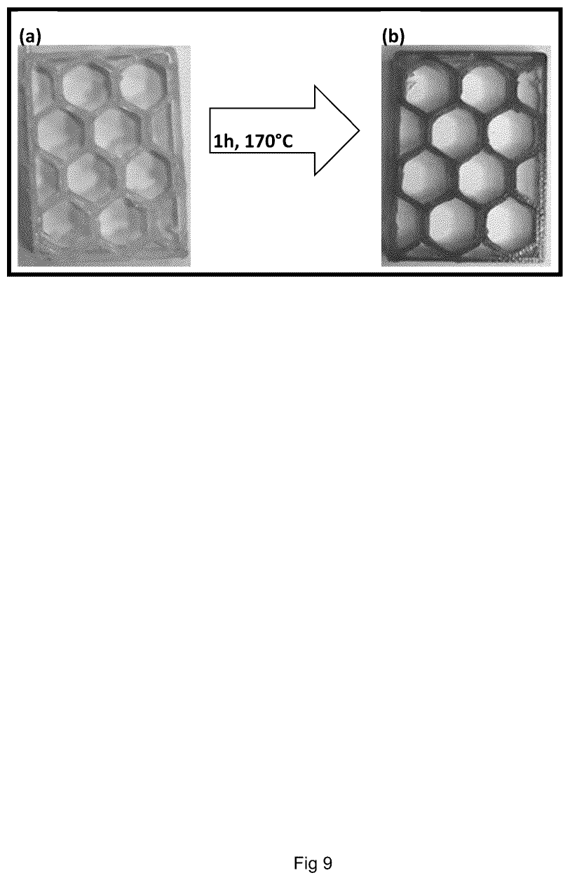

CHDO-PA-mea/meth was also 3D printed using a Hyrel System 30 M printer with an extrusion reservoir print head combined with a 356 nm UV lamp. A needle nozzle 20 gauge was used to deposit layers 0.45 mm in thickness. Extrusion occurred at room temperature on a print platform at room temperature at an extrusion speed of 1350 pulses/μm and the UV lamp continuously turned on providing the honeycomb structure in FIG. 9a. A curing step at 170° C. for 1 h was performed to consolidate the UV cured structure, leading to the material given in FIG. 9b.

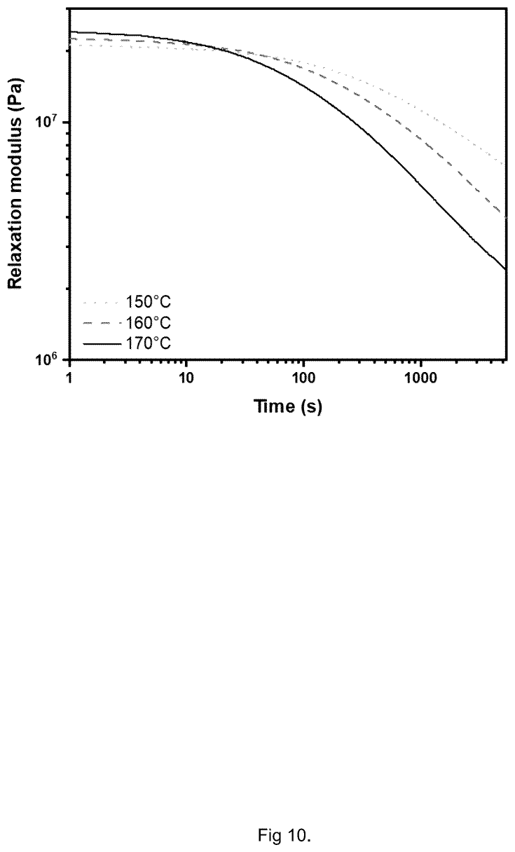

Viscoelastic properties of CHDO-PA-meth dual cured vitrimer were measured by stress relaxation experiments recorded on Anton Paar Physica MCR 302 rheometer in torsion mode at 1% shear strain (FIG. 10). The relaxation time of the polymer was clearly noticeable and recorded at 150° C., 160° C. and 170° C.

### Example 5: Synthesis of Benzoxazine Containing Free Aliphatic Groups, Ester Groups, and Methacrylate Groups from 1,4 Cyclohexanediol (CHDO) and 3-(4-hydroxyphenyl) Propionic Acid (PA) as Phenolic Acid Derivatives and Mono-Ethanolamine (Mea) as Primary Amine with Aliphatic OH, and Methacryloyl Chloride (MCI) as Methacrylate

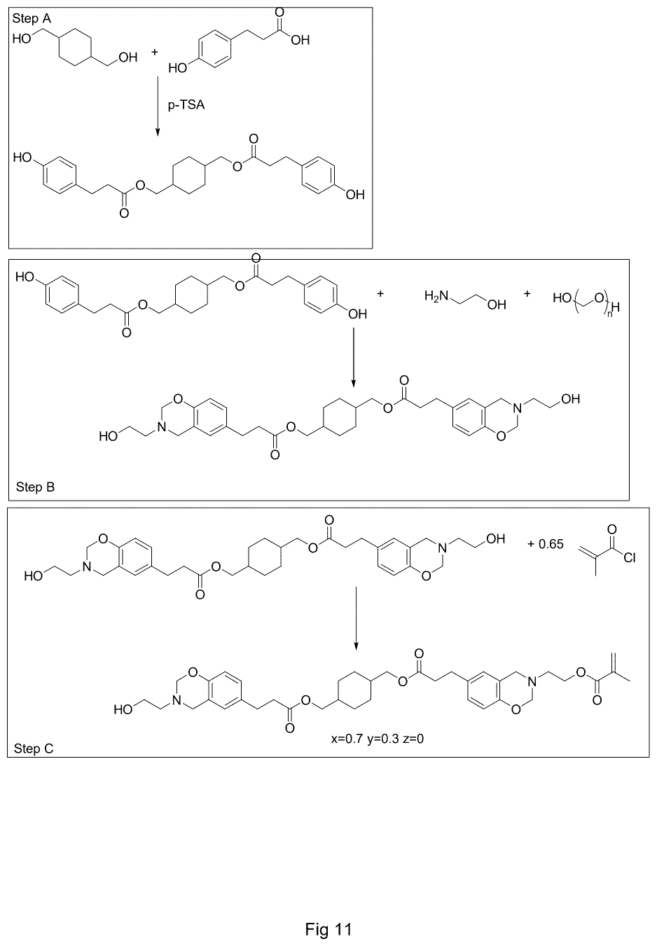

The CHDO-PA-mea/meth benzoxazine monomer containing free aliphatic hydroxyl groups and methacrylate groups was synthesized in three stages (FIG. 11). The first step, step a), corresponds to a Fischer esterification between 1,4 cyclohexanediol (cyclohexanediol, CHDO) (1 eq.) and 3-(4-hydroxyphenyl) propionic acid (phloretic acid, PA) (2 eq.) in presence of para-toluene sulfonic acid introduced in catalytic amount (0.5 wt. %). The reactants were put together in melt at 130° C. and agitated by mechanical stirring for 24 hours, to provide (CHDO-PA) (1 eq.).

The second step, step b), corresponds to a Mannich condensation of CHDO-PA (1 eq.) with mono-ethanolamine (mea) (2 eq.) and paraformaldehyde (PFA) (4 eq,). All these reactants were agitated together by mechanical stirring and reacted in melt at 70° C. for 8 hours (85° C. for 2 hours) to provide the CHDO-PA-mea benzoxazine containing free aliphatic hydroxyl groups.

In said example, the primary amine is omitted, the mono-ethanolamine (mea) is used as providing the both moieties (primary amine an amino-alcohol).

The third step, step c), corresponds to the methacrylation of CHDO-PA-mea with methacryloyl chloride (0.65 eq.). CHDO-PA-mea was dried overnight under reduced pressure to remove traces of water. It was thus solubilized in dried CH₂Cl₂. Triethylamine (TEA, 0.65 eq.) was added to the solution, as a catalyst, and the mixture was poured in a ice bath. Then, methacryloyl chloride was added drop-wise, and the solution recovered to room temperature and stirred overnight to provide CHDO-PA-mea/meth with z=0, x=0.7 and y=0.3.

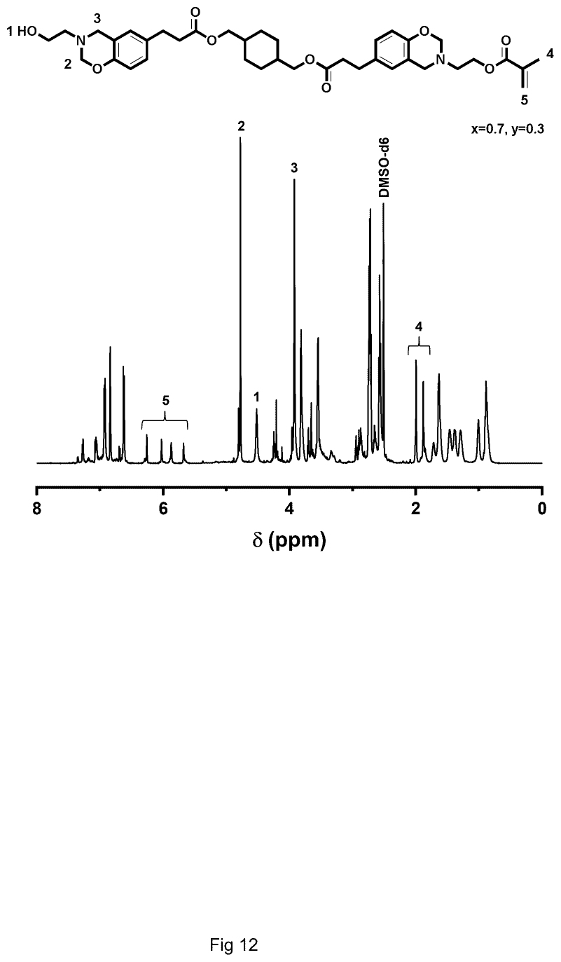

The FIG. 12 is displaying the ¹H NMR spectrum (AVANCE Ill HD Bruker spectrometer) of CHDO-PA-mea/meth ester-containing benzoxazine monomers where z=0, x=0.7 and y=0.3.

### Example 6. Two Step Synthesis Using CHDO-PA-Mea/Meth of Example 5

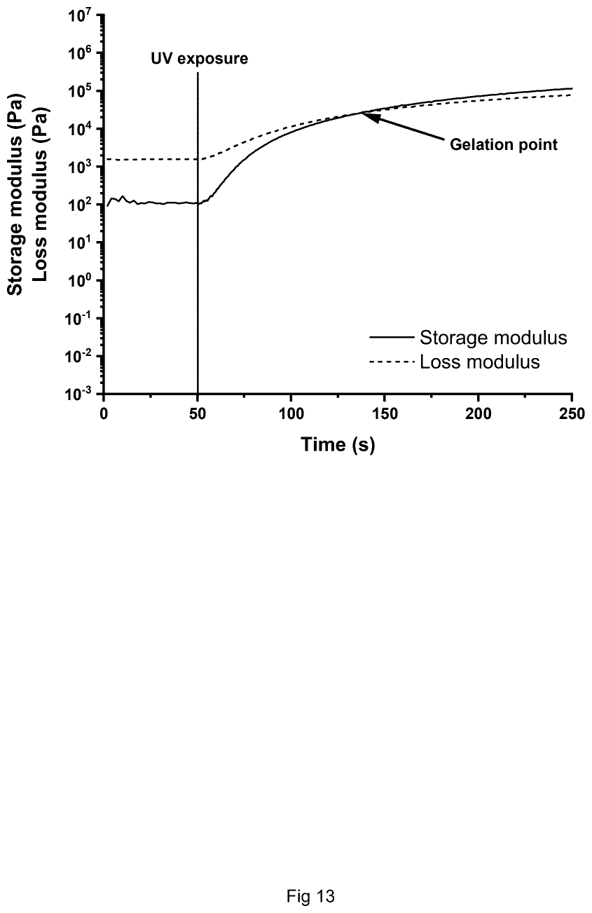

The UV curing of CHDO-PA-mea/meth of Example 5 was monitored by rheological measurement in FIG. 13. The rheogram is performed under the following conditions: 10 Hz, with constant amplitude of 0.1%; 25 mm plates. The test is performed by exposing UV light with a Omnicure device (power, wavelength), the gelation is reached after 84 seconds.

CHDO-PA-mea/meth was also 3D printed using a Hyrel System 30 M printer with an extrusion reservoir print head combined with a 356 nm UV lamp. A needle nozzle 20 gauge was used to deposit layers 0.45 mm in thickness. Extrusion occurred at room temperature on a print platform at room temperature at an extrusion speed of 1350 pulses/Jim and the UV lamp continuously turned on providing the honeycomb structure. However, the level of methacrylation was not high enough for the material to withstand during the curing step, leading to its collapse.

### Example 7: Synthesis of Benzoxazine Containing Free Aliphatic Groups, Ester Groups, and Methacrylate Groups from 1,2 Ethylenediol (ETDO) and 3-(4-hydroxyphenyl) Propionic Acid (PA) as Phenolic Acid Derivatives and Mono-Ethanolamine (Mea) as Primary Amine with Aliphatic OH, and Methacryloyl Chloride (MCI) as Methacrylate

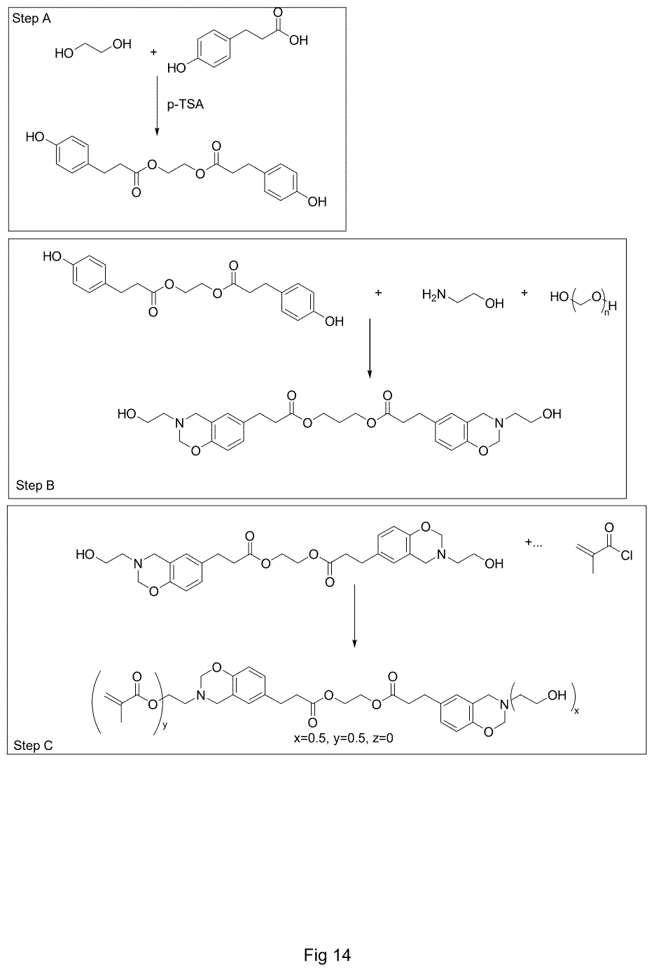

The ETDO-PA-mea/meth benzoxazine monomer containing free aliphatic hydroxyl groups and methacrylate groups was synthesized in three stages (FIG. 14). The first step, step a), corresponds to a Fischer esterification between 1,4 ethylenediol (ethylenediol, ETDO) (1 eq.) and 3-(4-hydroxyphenyl) propionic acid (phloretic acid, PA) (1 eq.) in presence of para-toluene sulfonic acid introduced in catalytic amount (0.5 wt. %). The reactants were put together in melt at 130° C. and agitated by mechanical stirring for 24 hours, to provide (ETDO-PA) (1 eq.).

The second step, step b), corresponds to a Mannich condensation of ETDO-PA (1 eq.) with mono-ethanolamine (mea) (2 eq.) and paraformaldehyde (PFA) (4 eq,). All these reactants were agitated together by mechanical stirring and reacted in melt at 70° C. for 8 hours to provide the ETDO-PA-mea benzoxazine containing free aliphatic hydroxyl groups.

In said example, the primary amine is not omitted, mono-ethanolamine (mea) and furfurylamine (fa) are used.

The third step, step c), corresponds to the methacrylation of ETDO-PA-mea with methacryloyl chloride (6 eq.). ETDO-PA-mea was dried overnight under reduced pressure to remove traces of water. It was thus solubilized in dried CH₂Cl₂. Triethylamine (TEA, 6 eq.) was added to the solution, as a catalyst, and the mixture was poured in a ice bath. Then, methacryloyl chloride was added drop-wise, and the solution recovered to room temperature and stirred overnight to provide ETDO-PA-mea/meth with z=0, x=0.5 and y=0.5.

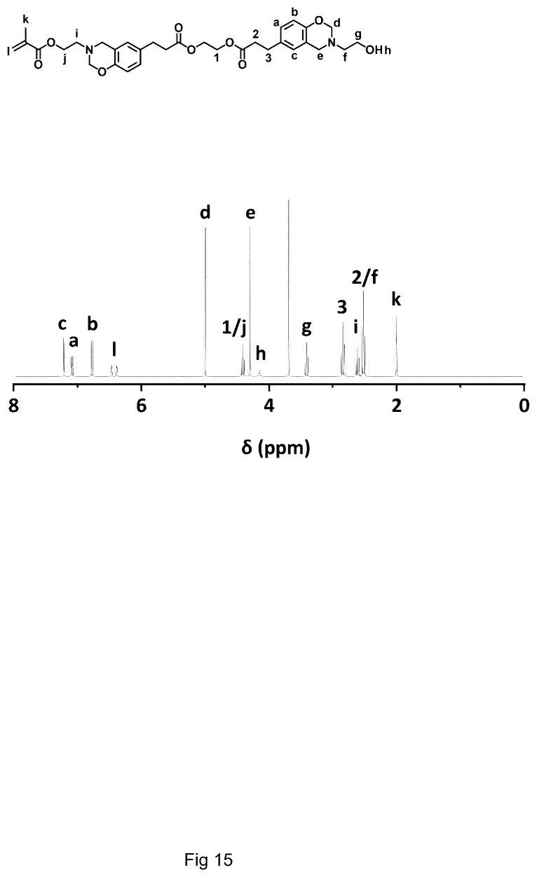

The FIG. 15 is displaying the ¹H NMR spectrum (AVANCE Ill HD Bruker spectrometer) of ETDO-PA-mea/meth ester-containing benzoxazine monomers where z=0, x=0.5 and y=0.5.

### Example 8: Synthesis of Benzoxazine Containing Free Aliphatic Groups, Ester Groups, and Methacrylate Groups from 1,2 Cyclohexanediol (CHDO) and 3-(4-hydroxyphenyl) Propionic Acid (PA) as Phenolic Acid Derivatives, Furfurylamine (Fa) as Primary Amine and Mono-Ethanolamine (Mea) as Primary Amine with Aliphatic OH, and Methacryloyl Chloride (MCI) as Methacrylate

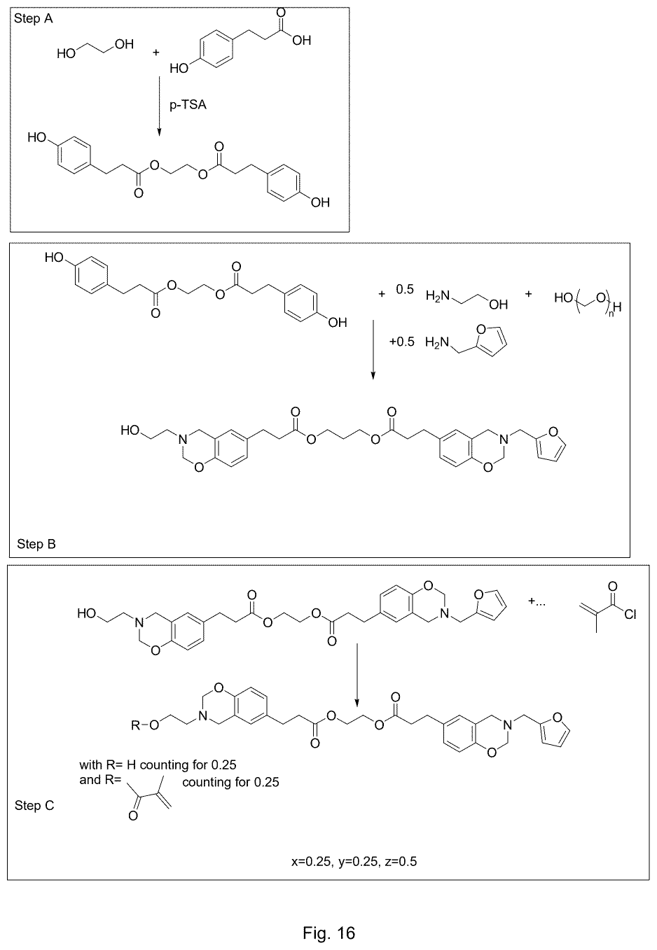

The CHDO-PA-mea/fa/meth benzoxazine monomer containing free aliphatic hydroxyl groups and methacrylate groups was synthesized in three stages (FIG. 16). The first step, step a), corresponds to a Fischer esterification between 1,4 cyclohexanediol (cyclohexanediol, CHDO) (1 eq.) and 3-(4-hydroxyphenyl) propionic acid (phloretic acid, PA) (1 eq.) in presence of para-toluene sulfonic acid introduced in catalytic amount (0.5 wt. %). The reactants were put together in melt at 130° C. and agitated by mechanical stirring for 24 hours, to provide (CHDO-PA) (1 eq.).

The second step, step b), corresponds to a Mannich condensation of CHDO-PA (1 eq.) with furfurylamine (fa) (0.5 eq) mono-ethanolamine (mea) (1.5 eq.) and paraformaldehyde (PFA) (4 eq,). All these reactants were agitated together by mechanical stirring and reacted in melt at 70° C. for 8 hours to provide the CHDO-PA-mea/fa benzoxazine containing free aliphatic hydroxyl groups.

The third step, step c), corresponds to the methacrylation of CHDO-PA-mea/fa with methacryloyl chloride (6 eq.). CHDO-PA-mea/fa was dried overnight under reduced pressure to remove traces of water. It was thus solubilized in dried CH₂Cl₂. Triethylamine (TEA, 6 eq.) was added to the solution, as a catalyst, and the mixture was poured in a ice bath. Then, methacryloyl chloride was added drop-wise, and the solution recovered to room temperature and stirred overnight to provide CHDO-PA-mea/fa/meth with z=0.5, x=0.25 and y=0.25.

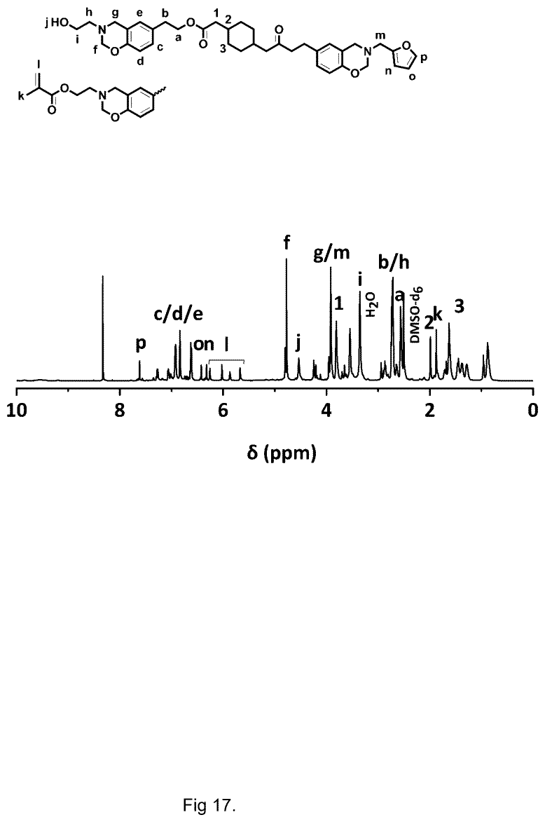

The FIG. 17 is displaying the ¹H NMR spectrum (AVANCE Ill HD Bruker spectrometer) of CHDO-PA-mea/fa/meth ester-containing benzoxazine monomers where z=0.5, x=0.25 and y=0.25.

### Example 9: (Comparative Example, not of the Invention)

Synthesis of benzoxazine not containing free aliphatic groups, but containing ester groups, and methacrylate groups from 1,4 cyclohexanediol (CHDO) and 3-(4-hydroxyphenyl) propionic acid (PA) as phenolic acid derivatives and mono-ethanolamine (mea) as primary amine with aliphatic OH, and methacryloyl chloride (MCI) as methacrylate.

Example 9 is a comparative example. In this molecule, all of the aliphatic —OH groups have been replaced with methacrylate groups. The molecule in question has the ability to be cured using UV light, but it lacks aliphatic —OH groups, which prevents it from undergoing any dynamic exchanges. As a result, the material cannot exhibit the properties of a vitrimer.

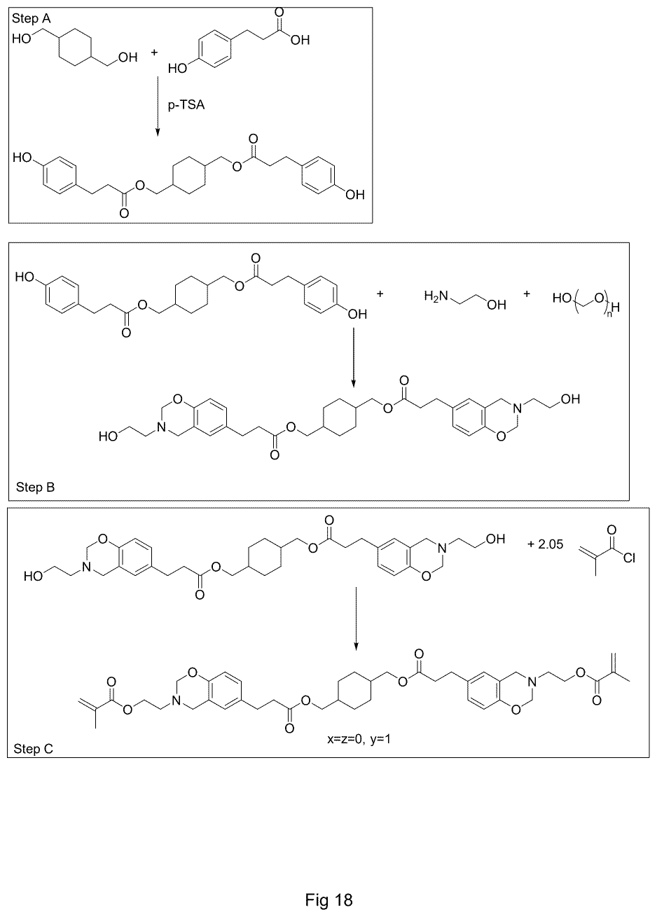

The CHDO-PA-meth benzoxazine monomer containing free aliphatic hydroxyl groups and methacrylate groups was synthesized in three stages (FIG. 18). The first step, step a), corresponds to a Fischer esterification between 1,4 cyclohexanediol (cyclohexanediol, CHDO) (1 eq.) and 3-(4-hydroxyphenyl) propionic acid (phloretic acid, PA) (2 eq.) in presence of para-toluene sulfonic acid introduced in catalytic amount (0.5 wt. %). The reactants were put together in melt at 130° C. and agitated by mechanical stirring for 24 hours, to provide (CHDO-PA) (1 eq.).

The second step, step b), corresponds to a Mannich condensation of CHDO-PA (1 eq.) with mono-ethanolamine (mea) (2 eq.) and paraformaldehyde (PFA) (4 eq,). All these reactants were agitated together by mechanical stirring and reacted in melt at 70° C. for 8 hours (85° C. for 2 hours) to provide the CHDO-PA-mea benzoxazine containing free aliphatic hydroxyl groups.

In said example, the primary amine is not omitted, the mono-ethanolamine (mea) is used as providing the both moieties (primary amine an amino-alcohol).

The third step, step c), corresponds to the methacrylation of CHDO-PA-mea with methacryloyl chloride (4 eq.). CHDO-PA-mea was dried overnight under reduced pressure to remove traces of water. It was thus solubilized in dried CH₂C₁₂. Triethylamine (TEA, 2.05 eq.) was added to the solution, as a catalyst, and the mixture was poured in a ice bath. Then, methacryloyl chloride was added drop-wise, and the solution recovered to room temperature and stirred overnight to provide CHDO-PA-mea/meth with x=z=0 and y=1.

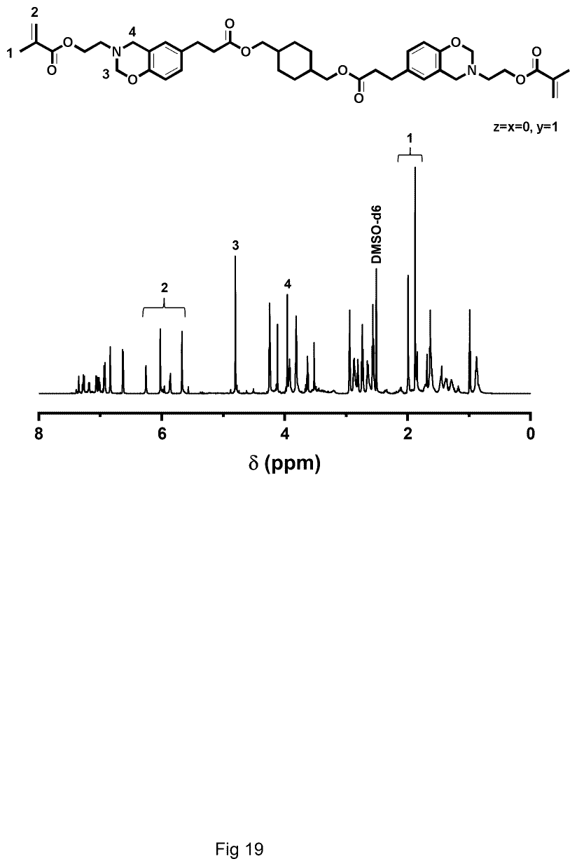

The FIG. 19 is displaying the ¹H NMR spectrum (AVANCE Ill HD Bruker spectrometer) of CHDO-PA-meth ester-containing benzoxazine monomers.

### Example 10. (Comparative Example)

Two Step Synthesis from CHDO-PA-Mea/Meth (x=z=0 and y=1)

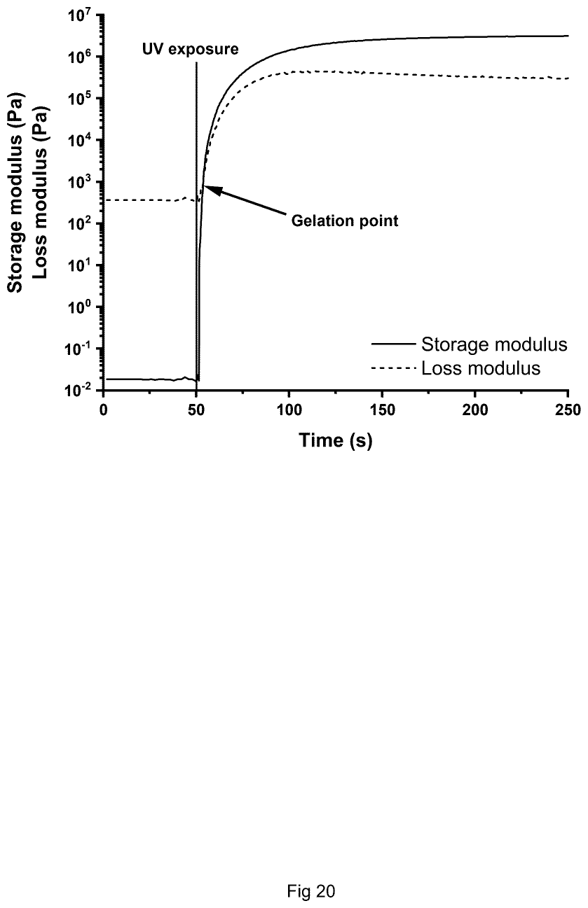

The UV curing of CHDO-PA-meth of Example 9 was monitored by rheological measurement in FIG. 20. The rheogram is performed under the following conditions: 10 Hz, with constant amplitude of 0.1%; 25 mm plates. The test is performed by exposing UV light with a Omnicure device (power, wavelength), the gelation is reached after 4 seconds.

Rectangular bars of size 25 mm*5 mm*1.5 mm were prepared by exposing the resin to UV curing during 5 minutes, and then subjected to thermal curing at 170° C. for 1 h.

Viscoelastic properties of CHDO-DPA-meth dual cured vitrimer were measured by stress relaxation experiments recorded on Anton Paar Physica MCR 302 rheometer in torsion mode at 1% shear strain. The polymer was not able to relax whatever the temperature, as it does not contain any aliphatic —OH groups able to dynamically exchange with the ester bonds.

1. An ester and acrylate moieties containing benzoxazine monomer of formula (I), 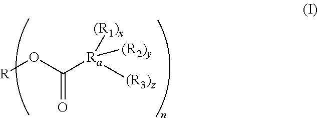
wherein
R₁ is 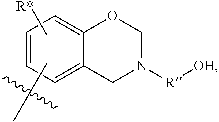
R₂ is 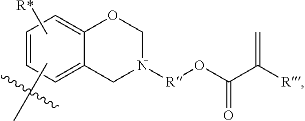
and
R₃ is 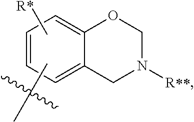
wherein
Rₐ is selected from the group consisting of a linear or branched C₁-C₆ alkyl or alkoxy group, a linear or branched C₂-C₆ alkenyl or alkylenoxy group, a substituted or unsubstituted linear or branched C₂-C₆ alkynyl group, and a —C-linear or branched C₁-C₆ alkyl or C₂-C₆ alkenyl substituted or unsubstituted phenyl group;
R* is selected from the group consisting of H, OH and a O-linear or branched C₁-C₆ alkyl group, a linear or branched C₁-C₁₅ alkyl group or a C₂-C₁₅ alkenyl group or 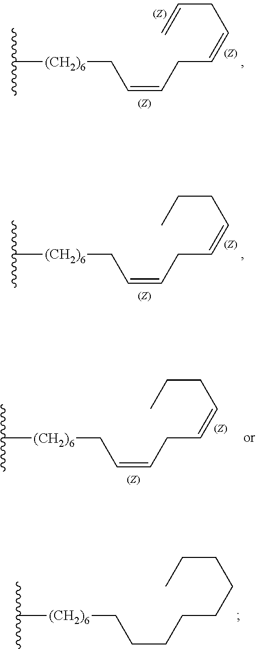
R″, R** and R are, independently, selected from the group consisting of a linear or branched C₁-C₆ alkyl or alkoxy group; a linear or branched C₂-C₆ alkenyl or alkylenoxy group; a substituted or unsubstituted linear or branched C₂-C₆ alkynyl group; at least one linear or branched C₁-C₆ alkyl or C₂-C₆ alkenyl substituted or unsubstituted o-, m-, p-phenyl group, cyclo(C₃-C₆alkyl) group or a heterocyclo(C₃-C₆alkyl) group, wherein the hetero atom is selected from N, S, and O; a (CH₂)ₙ₃-phenyl group, a —(CH₂)ₙ₁—O—(CH₂)ₙ₂ group, a —CHₙ₁((CHₙ₂OH)ₙ₁)CHₙ₁— group wherein n1 and n2, independently, are an integer
from 1 to 10, 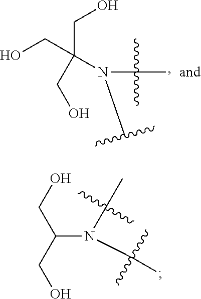
R′″ is Me, H or CN;
n3 is an integer 1-10;
with the proviso that
1≤n<50 and x>0, y>0 and z>0, and independently of n, x+y+z=1;
x, y, z represent the proportion between benzoxazine groups when prepared from an aminoalcohol and the other amine(s) and methacrylate groups,
$$x = \frac{{n}_{aminoalcohol}}{{n}_{functional groups}^{total}}$$
$$y = \frac{{n}_{acrylate}}{{n}_{functional groups}}$$
$$z = \frac{{n}_{amines}}{{n}_{functional groups}}$$
wherein
$${n}_{functional groups}^{total} = {n}_{aminoalcohol} + {n}_{amines} + {n}_{acrylate} , and {n}_{aminoalcohol}$$
being the number of aminoalcohol, nₐₘᵢₙₑₛ represent the number of amines (exception the number of aminoalcohol), n_{acrylate} corresponds to the number of acrylate/methacrylate groups, and
$${n}_{funtional groups}^{total}$$
is the total number of functional groups bore by the benzoxazine rings.

2. The monomer according to claim 1, wherein n is within the range of 1-10, and, independently, Ra is selected from the group consisting of a linear or branched C₁-C₄ alkyl or alkoxy group, a linear or branched C₂-C₄ alkenyl or alkylenoxy group, a substituted or unsubstituted linear or branched C₂-C₄ alkynyl group, and a linear or branched C₁-C₄ alkyl or C₂-C₆ alkenyl substituted or unsubstituted phenyl group; and, independently,
R* is selected from the group consisting of H, OH and a O-linear or branched C₁-C₄ alkyl group, a linear or branched C₁-C₁₀ alkyl group or a C₂-C₁₀ alkenyl group, more preferentially a linear or branched C₁-C₆ alkyl group, a C₂-C₆ alkenyl group or a C₂-C₆ alkynyl group or

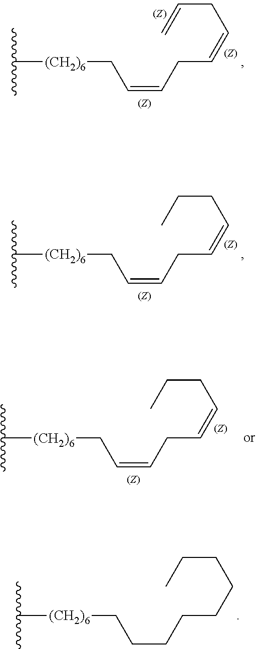

3. The monomer according to claim 1, wherein, in the definition of R″, each C atom, independently, of the phenyl group, the cyclo(C₃-C₆alkyl) group or the heterocyclo(C₃-C₆alkyl) group is bearing substituents as defined in claim 1 or 2.

4. A process for synthesizing an ester and acrylate moieties-containing benzoxazine monomer of formula (I), comprising:
a) reacting a phenolic carboxylic acid of formula (II), comprising at least one R* group on the phenolic ring: 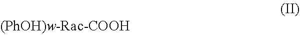
wherein
Rac has the definition of Ra and w=1, 2 or 3;
with a hydroxyl moiety bearing compound of formula (III) 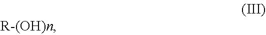
wherein R has the definition of R″ and n has the same definition than above, at a temperature of from 25° C. to 200° C., during 1 h-72 h, in the presence of a catalyst of Bronsted acid type and under inert atmospheric conditions, resulting in a phenol terminated oligomer or molecule (compound (IV),
b) reacting the compound (IV) with a mixture of:
an amino-alcohol of formula (V): 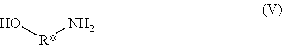
an aldehyde compound selected from formaldehyde and paraformaldehyde of formula 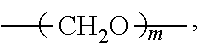
where m is an integer of from 8 to 100,
a primary amine of formula (VI), 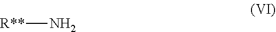
at a temperature of from 50° C. to 200° C., during 1 h-12 h, under inert atmospheric conditions, providing a compound of formula (VII), and
c) reacting the compound (VII) with a compound bearing acrylate or methacrylate groups, said compound being at least one defined by compounds (VIII)—(XII), (meth)acrylic acid chloride of formula (VIII) 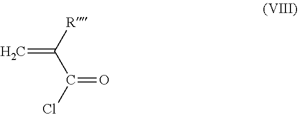
in polar solvents, at temperatures from −5° C. to 25° C.; or
methyl(meth)acrylate of formula (IX) 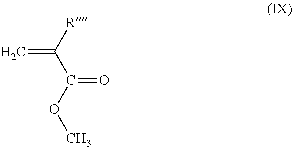
in the presence of a catalyst and a polymerization inhibitor; or
(meth)acrylic acid of formula (X) 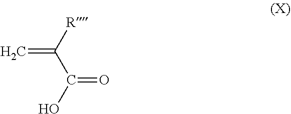
in the presence of a catalyst and an azodicarboxylate in a solvent at temperatures from −10° C. to reflux; or
an excess of (meth)acrylic acid of formula (X) in the presence of a catalyst; or
(meth)acrylic acid anhydride of formula (XI) or asymmetric anhydride of formula (XII) 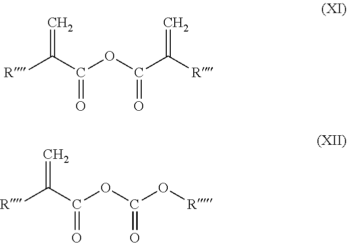
in the presence of a catalyst;
wherein
R″″ is H or CH₃.
R′″″ is CH₃ or C₂H₅;
at temperatures of from −10° C.-50° C., during 12 h-48 h, for obtaining the ester and acrylate moieties-containing benzoxazine monomer of formula (I), x, y, z, in formula (I), represent the proportion between benzoxazine groups when prepared from an aminoalcohol and the other amine(s) and methacrylate groups, the latter obtained after step c),
$$x = \frac{{n}_{aminoalcohol}}{{n}_{functional groups}^{total}}$$
$$y = \frac{{n}_{acrylate}}{{n}_{functional groups}}$$
$$z = \frac{{n}_{amines}}{{n}_{functional groups}}$$
wherein
$${n}_{functional groups}^{total} = {n}_{aminoalcohol} + {n}_{amines} + {n}_{acrylate} , and {n}_{aminoalcohol}$$
being the number of aminoalcohol, nₐₘᵢₙₑₛ represent the number of amines (exception the number of aminoalcohol), n_{acrylate} corresponds to the number of acrylate/methacrylate groups, and
$${n}_{funtional groups}^{total}$$
is the total number of functional groups bore by the benzoxazine rings,
with the proviso that when at least one R* of the phenolic acid derivative is in ortho position with regard to —OH group, then R* is H.
wherein the respective stoichiometry of starting reactants on step a), phenolic carboxylic acid:hydroxyl moiety bearing compound is n eq.:1.0 eq, resulting in an 1.0 eq. of the phenol terminated oligomer or molecule (compound (IV)), and
wherein the temperature range of step b) is of from 70° C. to 150° and step b) is performed from 1 h to 8 h.

5. The process according to claim 4, wherein the amino-alcohol of formula (V) includes R* group, a linear amino-alcohol with a primary amine moiety and an aliphatic hydroxyl moiety.

6. The process according to claim 4, wherein the primary amines are selected from the group consisting in allylamine, methylamine, ethylamine, propylamine, butylamine, isopropylamine, hexylamine, cyclohexylamine, stearylamine, 2-aminofluorene, aminophenyl acetylene, propargyl ether aniline, 4-aminobenzonitrile, furfurylamine and aniline, or mixtures thereof.

7. The process according to claim 4, wherein the respective stoichiometry of starting reactants on step b), phenol terminated oligomer or molecule (IV):amino-alcohol (V):primary amine (VI):paraformaldehyde is 1.0 eq.: n(x+y): nz: 2.0 n (x+y+z) resulting in an 1.0 eq. of compound (VII).

8. The process according to claim 4, wherein step c) is performed with compound (VIII) -(meth)acrylic acid chloride- via an acylation reaction with compound (VII) of step b), in the absence of any suitable catalyst or in the presence of a catalyst/hydrogen absorbent, selected from triethyl amine (TEA), pyridine, N,N-Diisopropylethylamine (DIPEA) and N—N-dimethylaniline, in polar solvents, selected from acetonitrile, chloroform, dichloromethane and N,N-dimethylformamide (DMF), at temperatures of from −5 to 15° C.

9. The process according to claim 4, wherein step c) is performed with compound (IX) -methyl(meth)acrylate- via a re-esterification reaction, in the presence of a catalyst and a polymerization inhibitor, with a simultaneous distillation of the resulting azeotropic mixture.

10. The process according to claim 4, wherein step c) is carried with compound (X) -(meth)acrylic acid- via a Mitsunobu reaction involving the presence of a catalyst, and an azodicarboxylate, in a solvent, at temperatures from −10° C. to reflux.

11. The process according to claim 4, wherein step c) is performed with an excess of (meth)acrylic acid- compound (X), via an esterification reaction in the presence of an acidic catalyst, being selected from p-toluenesulfonic acid (p-TCA), sulfuric acid (H₂SO₄) and methanesulfionic acid (CF₃SO₃H), with simultaneous distillation of the resulting azeotropic mixture.

12. The process according to claim 4, wherein step c) is performed with compound (XI) -(meth)acrylic acid anhydride- or with compound (XII) -(meth)acrylic acid asymmetric anhydride-, via an acylation reaction in the presence of a catalyst, being selected from p-toluenesulfonic acid (p-TCA), sulfuric acid (H₂SO₄) and anhydrous ZnCl₂, at temperature range of −5° C. to 15° C.

13. The process according to claim 4, wherein the respective stoichiometry of starting reactants on step c), compound VII: compound bearing acrylate or methacrylate groups (VIII-XII) is 1.0 eq.:3.0 ny eq. resulting in an 1.0 eq. of the ester and acrylate moieties-containing benzoxazine monomer,

14. A process for preparing a polybenzoxazine derivative vitrimer comprising a first curing step by UV treatment polymerization of an ester and acrylate moieties-containing benzoxazine monomer of formula (I) as defined in claim 1, followed by a second curing step performed by a thermic treatment undergoing the polymerisation of the benzoxazine moieties of the ester and acrylate moieties-containing benzoxazine monomer(s).

15. The process according to claim 14, wherein the first curing step is carried out with the presence of a radical initiator, at % weights (% wt) that are of from 1 wt % to 5 wt %.

16. The process according to claim 14, wherein the second curing step is carried out at temperature ranges of from 100° C. to 250° C., for 1 h-10 h.

17. A polybenzoxazine derivative vitrimer, that is obtainable by the process of claim 14, exhibiting at least one of the following characteristics:
(i) Tᵥ values of from 100° C. to 250° C., and
(ii) Relaxation temperature values, ≥Tᵥ values, of from 100° C. to 300° C.

18. The process according to claim 14, wherein the ester and acrylate moieties-containing benzoxazine monomer of formula (I), are synthesized by a process comprising:
a) reacting a phenolic carboxylic acid of formula (II), comprising at least one R* 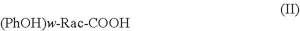
wherein
Rac has the definition of Ra and w=1, 2 or 3;
with a hydroxyl moiety bearing compound of formula (III) 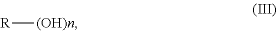
wherein R has the definition of R″ and n has the same definition than above, at a temperature of from 25° C. to 200° C., during 1 h-72 h, in the presence of a catalyst of Bronsted acid type and under inert atmospheric conditions, resulting in a phenol terminated oligomer or molecule (compound (IV),
b) reacting the compound (IV) with a mixture of:
an amino-alcohol of formula (V): 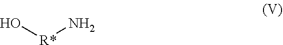
an aldehyde compound selected from formaldehyde and paraformaldehyde of formula 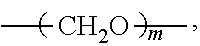
where m is an integer of from 8 to 100,
a primary amine of formula (VI), 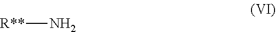
at a temperature of from 50° C. to 200° C., during 1 h-12 h, under inert atmospheric conditions, providing a compound of formula (VII), and
c) reacting the compound (VII) with a compound bearing acrylate or methacrylate groups, said compound being at least one defined by compounds (VIII)—(XII),
(meth)acrylic acid chloride of formula (VIII) 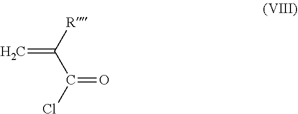
in polar solvents, at temperatures from −5° C. to 25° C.; or
methyl(meth)acrylate of formula (IX) 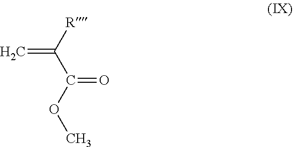
in the presence of a catalyst and a polymerization inhibitor; or
(meth)acrylic acid of formula (X) 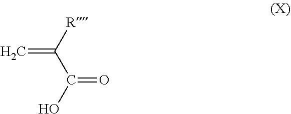
in the presence of a catalyst and an azodicarboxylate in a solvent at temperatures from −10° C. to reflux; or
an excess of (meth)acrylic acid of formula (X) in the presence of a catalyst;
or
(meth)acrylic acid anhydride of formula (XI) or asymmetric anhydride of formula (XII) 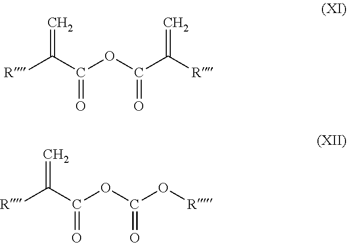
in the presence of a catalyst;
wherein
R″″ is H or CH₃.
R′″″ is CH₃ or C₂H₅;
at temperatures of from −10° C.-50° C., during 12 h-48 h, for obtaining the ester and acrylate moieties-containing benzoxazine monomer of formula (I), x, y, z, in formula (I), represent the proportion between benzoxazine groups when prepared from an aminoalcohol and the other amine(s) and methacrylate groups, the latter obtained after step c),
$$x = \frac{{n}_{aminoalcohol}}{{n}_{functional groups}^{total}}$$
$$y = \frac{{n}_{acrylate}}{{n}_{functional groups}}$$
$$z = \frac{{n}_{amines}}{{n}_{functional groups}}$$
wherein
$${n}_{functional groups}^{total} = {n}_{aminoalcohol} + {n}_{amines} + {n}_{acrylate} , and {n}_{aminoalcohol}$$
being the number of aminoalcohol, nₐₘᵢₙₑₛ represent the number of amines (exception the number of aminoalcohol), n_{acrylate} corresponds to the number of acrylate/methacrylate groups, and
$${n}_{funtional groups}^{total}$$
is the total number of functional groups bore by the benzoxazine rings,
with the proviso that when at least one R* of the phenolic acid derivative is in ortho position with regard to —OH group, then R* is H.
wherein the respective stoichiometry of starting reactants on step a), phenolic carboxylic acid:hydroxyl moiety bearing compound is n eq.:1.0 eq, resulting in an 1.0 eq. of the phenol terminated oligomer or molecule (compound (IV)), and
wherein the temperature range of step b) is of from 70° C. to 150° and step b) is performed from 1 h to 8 h.
